(19) United States
(12) Patent Application Publication
(10) Pub. No.: US 2025/0073004 A1
(43) Pub. Date: Mar. 6, 2025

(54) NEURAL REPRESENTATION OF ORAL DATA

(71) Applicant: Align Technology, Inc., San Jose, CA (US)

(72) Inventors: Nicolas Wicki, Zürich (CH); Janik Lobsiger, Zürich (CH); Philipp Kopp, Zürich (CH); Sinan Ibrahim Bayraktar, Zürich (CH); Michael Chang, Zürich (CH); Niko Benjamin Huber, Zürich (CH)

(21) Appl. No.: 18/821,445

(22) Filed: Aug. 30, 2024

Related U.S. Application Data
(60) Provisional application No. 63/535,502, filed on Aug. 30, 2023.

(51) Int. Cl. A61C 9/00; A61B 5/00; G06T 7/00; G06T 17/00

(52) U.S. Cl. CPC A61C 9/0053; A61B 5/4547; G06T 7/0012; G06T 17/00; G06T 2207/10081; G06T 2207/30036

(57) ABSTRACT

A method includes obtaining first data of a dental arch having one or more first properties. The method further includes processing the first data using one or more trained machine learning models. The one or more trained machine learning models generate a dimensionally reduced representation of the dental arch based on the first data. The one or more trained machine learning models generate second data of the dental arch that has one or more second properties. The method further includes obtaining, from the one or more trained machine learning models, the second data of the dental arch that has the one or more second properties. The second data is based on the dimensionally reduced representation. The one or more second properties are different from the one or more first properties. The method further includes causing a representation of the dental arch to be displayed based on the second data.

### RELATED APPLICATIONS

This application claims the benefit of U.S. Provisional Patent Application No. 63/535,502, filed Aug. 30, 2023, entitled “NEURAL REPRESENTATION OF DENTAL ARCH DATA,” which is incorporated by reference herein.

### TECHNICAL FIELD

Embodiments of the present invention relate to the field of dentistry, and in particular to the generation of oral data for use for a dental patient.

### BACKGROUND

When a dentist or orthodontist is engaging with current and/or potential patients, it is often helpful to generate data indicative of dental arches of the patients. For example, it may be helpful to show those patients images of before and after treatments of previous patients with similar malocclusions who have undergone successful treatment. However, often those previous patients look very different from the current patient. Such differences can make it difficult for the current or potential patient to properly visualize how they might look after successful treatment due to these differences. The more differences there are between the current patient and the prior patients whose images or models are shown, the more distracting those differences can become, which detract from the current patient's ability to visualize themselves with similar corrected teeth.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method includes obtaining first data of a dental arch. The first data of the dental arch has one or more first properties. The method further includes processing the first data using one or more trained machine learning models. The one or more trained machine learning models generate a dimensionally reduced representation of the dental arch based on the first data. The one or more trained machine learning models generate second data of the dental arch that has one or more second properties. The method further includes obtaining, from the one or more trained machine learning models, the second data of the dental arch that has the one or more second properties. The second data is based on the dimensionally reduced representation. The one or more second properties are different from the one or more first properties. The method further includes causing a representation of the dental arch to be displayed based on the second data.

In another aspect of the present disclosure, a method includes obtaining first data of a dental arch, the first data corresponding to a first imaging technique. The method further includes providing the first data to a first trained machine learning model. The first trained machine learning model generates a dimensionally reduced representation of the dental arch based on the first data. The method further includes obtaining second data of the dental arch. The second data is based on the dimensionally reduced representation. The second data corresponds to a second imaging technique. The method further includes causing the second data to be displayed.

In another aspect of the present disclosure, a method includes obtaining first data of a dental arch having one or more first properties. The method further includes training a first machine learning model based on the first data. Training the machine learning model includes obtaining, by the first machine learning model, the first data. Training the machine learning model further includes reducing, by the first machine learning model, dimensionality of the first data to generate first compressed data. Training the machine learning model further includes generating first reconstructed data based on the first compressed data. Training the machine learning model further includes causing parameters of the first machine learning model to be adjusted based on one or more differences between the first data and the first reconstructed data.

### BRIEF DESCRIPTION OF THE DRAWINGS

The present disclosure is illustrated by way of example, and not by way of limitation in the figures of the accompanying drawings.

FIG. 1 is a block diagram illustrating an exemplary system architecture, according to some embodiments.

FIG. 2 illustrates a model training workflow and a model application workflow, according to some embodiments.

FIG. 3A is a diagram depicting operations of a model that is configured to reduce dimensionality of input data, according to some embodiments.

FIG. 3B is an example machine learning encoder for mapping dental arch data to a latent space, according to some embodiments.

FIG. 3C depicts operations for training or operating a model for mapping data representing dental arches, according to some embodiments.

FIG. 3D depicts operations of an autoencoder for generating dental arch data, according to some embodiments.

FIG. 3E depicts example operations of a dental arch data generator, according to some embodiments.

FIG. 3F depicts operations of a dental arch data generator in generating jaw pair data based on incomplete jaw pair data, according to some embodiments.

FIG. 3G depicts operations of a dental arch generator in generating dental arch data based on incomplete dental arch data, according to some embodiments.

FIG. 3H depicts operations of a dental arch data generator in generating dental arch data with different properties than provided input data, according to some embodiments.

FIG. 3I depicts operations of a dental arch data generator in generating treatment predictions, according to some embodiments.

FIG. 3J depicts example operations of a dental arch data generator in generating treatment predictions, according to some embodiments.

FIG. 3K depicts example operations of a dental arch data generator in generating oral structure predictions, according to some embodiments.

FIG. 3L depicts example operations of a dental arch data generator in generating oral structure predictions, according to some embodiments.

FIG. 3M depicts example operations of a dental arch data generator in predicting oral structures, according to some embodiments.

FIG. 3N depicts operations of a multi-stage treatment prediction platform, according to some embodiments.

FIG. 4A is a flow diagram of a method for generating a dataset for a machine learning model, according to some embodiments.

FIG. 4B is a flow diagram of a method for determining predictive dental arch data, according to some embodiments.

FIG. 4C is a flow diagram of a method for generating predictive dental arch data, according to some embodiments.

FIG. 4D is a flow diagram of a method for training a machine learning model for reducing dimensionality of dental arch data and generating synthetic dental arch data from the reduced dimensionality data, according to some embodiments.

FIG. 4E is a flow diagram of a method for determining predictive oral cavity data, according to some embodiments.

FIG. 4F is a flow diagram of a method for generating predictive oral cavity data, according to some embodiments.

FIG. 4G is a flow diagram of a method for training a machine learning model for reducing dimensionality of oral cavity data, according to some embodiments.

FIG. 5A illustrates a tooth repositioning system including a plurality of appliances, in accordance with some embodiments.

FIG. 5B illustrates a method of orthodontic treatment using a plurality of appliances, in accordance with some embodiments.

FIG. 6 illustrates a method for designing an orthodontic appliance to be produced by direct fabrication, in accordance with some embodiments.

FIG. 7 illustrates a method for digitally planning an orthodontic treatment and/or design or fabrication of an appliance, in accordance with some embodiments.

FIG. 8 is a block diagram illustrating a computer system, according to some embodiments.

### DETAILED DESCRIPTION

Described herein are technologies related to generating and utilizing a latent space representing dental arches. The latent space may be utilized for clinical or non-clinical applications. The latent space may be associated with a jaw pair measurement technique. The latent space may include representations of many variations of dental arches and/or jaw pairs. Dental arches or jaw pairs that are, in some way, similar to each other may be located nearby in the latent space, e.g., via a distance metric, cosine distance, or another similarity measure in the latent space. Representation of jaw pairs in a latent space may be utilized in making predictions of dental arch evolution, reducing dimensionality of dental arch data, generating adjusted dental arch data, augmenting sparse dental arch data, or the like.

Dental arch data may be utilized in treatment of a dental arch. For example, one or more dental malocclusions (e.g., misalignment of teeth) may be treated by an orthodontic treatment plan, which may include collecting and utilizing jaw pair data of a patient. As a further example, generation of a crown or dental implant may be performed based on dental arch data. Dental arch data may include data of one or more teeth (e.g., including size, shape, positioning, orientation, etc.), a group of teeth, an arch, an upper and lower jaw, etc.

In some systems, three-dimensional scans of dental arches may be utilized for dental treatments. Three-dimensional scans may include rich, detailed data, which may be well suited for generating a treatment plan, predicting treatment results, predicting shapes of missing teeth, or the like. Three-dimensional scans may include a large amount of data, be inconvenient to work with, may cause many computing operations to be performed, may occupy a large amount of communication bandwidth, etc. Further, predicting properties of absent portions of a jaw pair (e.g., missing teeth) from three-dimensional scans may include utilizing incomplete rule-based modeling, or depend on input from subject matter experts, which may be costly in terms of time, expertise, etc.

In some systems, two-dimensional images of jaw pairs may be utilized for dental treatments. For example, images of a patient smile may be utilized for predicting the appearance of a patient smile after dental treatment. Manipulating two-dimensional image data may provide an incomplete picture of dental treatment. For example, effects of teeth, surfaces, tissues, or the like obscured from the view of the camera may be difficult to ascertain from two-dimensional image data.

Methods and systems of the present disclosure may address one or more of the shortcomings of conventional systems. In some embodiments, a latent space representing dental arches, oral structures (e.g., including tissues such as gingiva and palate), and/or jaw pairs is generated. Any structures of an oral cavity (e.g., the mouth, including cheeks, tongue, upper and lower gums/gingiva, floor of the mouth, roof of the mouth, glands, etc.) may be represented in one or more latent spaces. The latent space may be a dimensionally reduced space that includes representation of dental arches and/or oral structures of an oral cavity. The latent space may be a dimensionally reduced space that includes regions representing a wide variety of dental arches and other oral structures. For example, healthy dental arches, dental arches requiring orthodontic treatment, dental arches including malocclusion or teeth misalignment, healthy or malformed gingiva, an upper palate, etc., may all be represented in the latent space associated with oral structures, e.g., including dental arches.

A machine learning model may be trained that maps data indicative of jaw pairs (e.g., data of one or more teeth, groups of teeth, arches, jaws, gingiva, the palate, or the like) to the latent space. Mapping may be performed based on complete oral data, e.g., complete oral scan data. Mapping may be performed based on incomplete data. For example, data including a subset of teeth of the dental arch, oral tissues without one or more teeth, sparse data, measurement data of a dental arch missing one or more teeth, etc., may be mapped to the latent space. The mapping may predict additional properties of the dental arch that are not captured in the measurement data, e.g., by completing the incomplete data.

In some embodiments, a machine learning model may map three dimensional oral scans (which may include teeth and other tissues, also referred to as intraoral scans) or a three dimensional model generated from intraoral scans to a latent space representing jaw pairs, oral data, or dental arches. In some embodiments, a machine learning model may map one or more two dimensional images (e.g., captured by a camera) to a latent space representing jaw pairs (e.g., including associated teeth and tissues) or dental arches. In some embodiments, a machine learning model may map data from different measurement techniques (e.g., computed tomography, x-ray, or other measurement techniques) to a latent space representing jaw pairs or dental arches.

In some embodiments, the mapping to the latent space may include a dimensional reduction of the input data. In some embodiments, the mapping to the latent space may be performed by an encoder model. In some embodiments, the mapping to the latent space may map jaw pairs that are similar (by one or more metrics) to similar locations (by one or more distance metrics) in the latent space.

In some embodiments, a machine learning model may map from a latent space representing dental arches and/or other oral data (e.g., compressed or dimensionally reduced representations of dental arches) to high dimensional data. The high dimensional data may resemble input data that is mapped to the latent space. For example, a machine learning model may map three-dimensional scans or a 3D model of dental arches to a latent space, and a second machine learning model may map the latent space representation to three-dimensional scan data or a 3D model of a dental arch. In some embodiments, another type of model may be utilized in mapping input data to the latent space, such as a statistical model. In some embodiments, numerical optimization methods may be utilized in mapping inputs to the latent space. A machine learning model may map from the latent space representing dental arches to three-dimensional representations of dental arches, two-dimensional images of dental arches, computed tomography images of dental arches, x-ray images of dental arches, etc.

In some embodiments, one or more transformations between and/or within a latent space may be performed. Transformations may be associated with transformations of a jaw pair or dental arch. For example, an orthodontic treatment may be represented by a transformation in the latent space. A full treatment may be represented by a transformation in the latent space. A stage of treatment may be represented by a transformation in the latent space. Transformations between latent spaces may be performed. For example, multiple latent spaces may be constructed that include representations of dental arches, and mapping between latent spaces may be utilized for predictions of jaw pair or dental arch data, transforming between data resembling different types of measurement instruments, etc. For example, a latent space may be utilized to approximate a three-dimensional scan of a jaw pair or dental arch based on one or more images of the jaw pair or dental arch. Further, a latent space may be utilized to approximate a scan including tissues (e.g., gingiva, palate) from three dimensional data of one or more teeth associated with the tissues. In some embodiments, transformations within a latent space may be performed by a machine learning model. In some embodiments, the latent space may be related to an autoencoder machine learning model. Compressing jaw pair or dental arch data to the latent space may be performed by an encoder, e.g., of an autoencoder. Expansion of latent space data to predicted jaw pair or dental arch data may be performed by a decoder, e.g., of an autoencoder.

Methods and systems of the present disclosure provide technological advantages over conventional systems. A latent space that provides a dimensionally reduced representation of dental arches and/or other oral data enables a compressed representation of dental arches, for clinical and non-clinical applications. A compressed representation enables a reduction in data storage space for storing data indicative of a jaw pair. A compressed representation enables communication of data indicative of a jaw pair while occupying less communication bandwidth than an uncompressed representation. A compressed representation enables operations to be performed on the representation with fewer computer operations, increasing computing efficiency and/or reducing computational expense of performing operations on jaw pair representation data.

A latent space representing dental arches and/or other oral data may enable operations based on sparse jaw pair or dental arch data. Sparse data may be of different forms. For example, data may be sparse due to one or more teeth missing from a jaw pair representation. Missing teeth may be due to data not including a representation of one or more teeth (for example, a two-dimensional image of a patient's face may not include representations of teeth positioned in the rear of the patient's mouth). Missing teeth of the jaw pair or dental arch data may be due to teeth missing from the patient's jaw pair or dental arch. Sparse data may be related to sparse data representation of a jaw pair or dental arch. For example, an incomplete three-dimensional scan may still be mapped to a latent space representing dental arches. A point cloud of one or more intraoral scans of a jaw pair or dental arch may be insufficient to act as a full scan set (e.g., insufficient to convert to one or more meshes representing a jaw pair or dental arch) but may map to a latent space representing one or more portions of dental arches.

Methods and systems of the present disclosure enable transformation of jaw pair and/or dental arch data collected via one measurement to a format of a different measurement. For example, predicted data of a different measurement type may be generated via one or more latent spaces representing dental arches. Transformations may be performed to data resembling data taken by different measurement equipment. Transformations may be performed to data resembling data taken by different measurement techniques. For example, predicted data may be generated via a latent space of three-dimensional intraoral scans, two-dimensional images, x-ray images, computed tomography images, two-dimensional segmentations or partitions of three-dimensional data, etc. Further, properties that are not collected in an initial measurement may be predicted via methods of the present disclosure. For example, predicted color data (e.g., texture maps including coloring information) may be generated based on input data that does not include color information, such as x-ray images. Predictive data representing dental arches and/or additional oral data may be generated without performing expensive, time-consuming, difficult, or inconvenient measurements. Predictive data may be utilized for further analysis, treatment planning, treatment progress checking, etc. Predictive data may be provided to further models, e.g., models that take as input measurement data that has not been collected for a target jaw pair. Predictive data may be utilized in reducing a number of performed computer operations. For example, a conventional three-dimensional scan generation may include multiple operations (generation of a point cloud, conversion to a mesh, conversion to a textured model, or the like). A latent space mapping may be performed directly from a point cloud, reducing the number of operations to generate the final three-dimensional model of the jaw pair or dental arch(es).

In one aspect of the present disclosure, a method includes obtaining first data of a dental arch. The first data of the dental arch has one or more first properties. The method further includes processing the first data using one or more trained machine learning models. The one or more trained machine learning models generate a dimensionally reduced representation of the dental arch based on the first data. The one or more trained machine learning models generate second data of the dental arch that has one or more second properties. The method further includes obtaining, from the one or more trained machine learning models, the second data of the dental arch that has the one or more second properties. The second data is based on the dimensionally reduced representation. The one or more second properties are different from the one or more first properties. The method further includes causing a representation of the dental arch to be displayed based on the second data.

In another aspect of the present disclosure, a method includes obtaining first data of a dental arch, the first data corresponding to a first imaging technique. The method further includes providing the first data to a first trained machine learning model. The first trained machine learning model generates a dimensionally reduced representation of the dental arch based on the first data. The method further includes obtaining second data of the dental arch. The second data is based on the dimensionally reduced representation. The second data corresponds to a second imaging technique. The method further includes causing the second data to be displayed.

In another aspect of the present disclosure, a method includes obtaining first data of a dental arch having one or more first properties. The method further includes training a first machine learning model based on the first data. Training the machine learning model includes obtaining, by the first machine learning model, the first data. Training the machine learning model further includes reducing, by the first machine learning model, dimensionality of the first data to generate first compressed data. Training the machine learning model further includes generating first reconstructed data based on the first compressed data. Training the machine learning model further includes causing parameters of the first machine learning model to be adjusted based on one or more differences between the first data and the first reconstructed data.

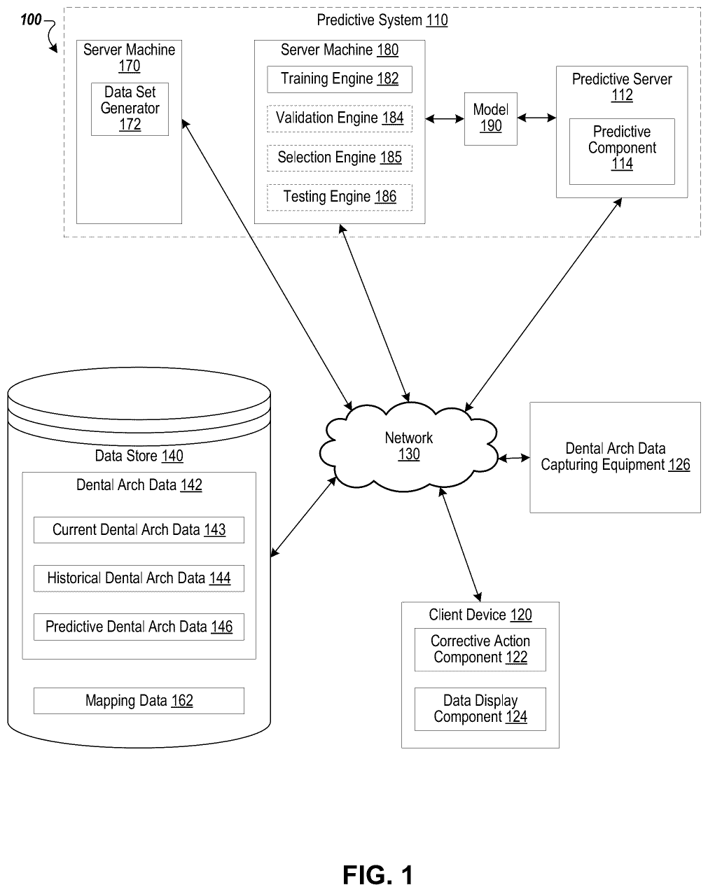

FIG. 1 is a block diagram illustrating an exemplary system 100 (exemplary system architecture), according to some embodiments. The system 100 includes a client device 120, dental arch data capturing equipment 126, predictive server 112, and data store 140. The predictive server 112 may be part of predictive system 110. Predictive system 110 may further include server machines 170 and 180. As used herein, when appropriate, techniques, operations, or components related to dental arches and dental arch data may be extended to include operations related to other oral structures, including upper and/or lower gingiva and palate. For example, dental arch data capturing equipment 126 may further be used for performing intraoral scans including generating data of other oral structures in addition to teeth.

Dental arch data capturing equipment 126 may provide dental arch data 142 (e.g., data indicative of oral structures, including current dental arch data 143, historical dental arch data 144). Dental arch data 142 may include data provided by any form of measurement associated with dental arches (e.g., three-dimensional intraoral scans, 3D models of dental arches and/or other oral structures generated from intraoral scans, 2D images, x-ray images, and so on). Dental arch data 142 may include data of one or more teeth. Dental arch data 142 may include data of a group or set of teeth. Dental arch data 142 may include data of an arch (e.g., an arch including or not including one or more teeth). Dental arch data 142 may include data of one or more jaws. Dental arch data 142 may include data of a jaw pair including an upper dental arch and a lower dental arch. Dental arch data 142 may include data of an upper arch and lower arch, comprising a jaw pair. Dental arch data 142 may include palatal data. Dental arch data 142 may include upper and/or lower gingiva data. Dental arch data 142 may be of different types. Dental arch data 142 may be or include two-dimensional images including dental arches or associated structures, e.g., teeth, jaws, etc. Dental arch data 142 may include two-dimensional images of faces or portions of faces including one or more teeth or other information of dental arches. Dental arch data 142 may include three-dimensional scans of dental arches, including completed (e.g., textured) scans, meshes, point clouds, or the like. Dental arch data 142 may include x-ray data. Dental arch data 142 may include computed tomography data. Dental arch data 142 may include segmentation data, e.g., two-dimensional partitions of three-dimensional data. Dental arch data capturing equipment 126 may include any combination of equipment for collecting dental arch data 142 (e.g., historical dental arch data 144), examples of which include intraoral scanners, x-ray machines, cameras, and so on. In some embodiments, dental arch data capturing equipment 126 corresponds to an intraoral scanner as described in U.S. Publication No. 2019/0388193, filed Jun. 19, 2019, entitled “Intraoral 3D Scanner Employing Multiple Miniature Cameras and Multiple Miniature Pattern Projectors,” which is incorporated by reference herein. In some embodiments, dental arch data capturing equipment 126 corresponds to an intraoral scanner as described in U.S. application Ser. No. 16/910,042, filed Jun. 23, 2020 and entitled “Intraoral 3D Scanner Employing Multiple Miniature Cameras and Multiple Miniature Pattern Projectors,” which is incorporated by reference herein. In some embodiments, dental arch data capturing equipment 126 corresponds to an intraoral scanner as described in U.S. Pat. No. 10,835,128, issued Nov. 17, 2020, which is incorporated by reference herein. In some embodiments, dental arch data capturing equipment 126 corresponds to an intraoral scanner as described in U.S. Pat. No. 10,918,286, issued Feb. 21, 2021, which is incorporated by reference herein. Dental arch data 142 may include data of healthy dental arches, dental arches including malocclusion or teeth misalignment, etc.

In some embodiments, dental arch data 142 may be processed (e.g., by the client device 120 and/or by the predictive server 112). Processing of the dental arch data 142 may include generating features. In some embodiments, the features are a pattern in the dental arch data 142 (e.g., slope, width, height, peak, etc.) or a combination of values from the dental arch data 142. Dental arch data 142 may include features and the features may be used by predictive component 114 for performing signal processing and/or for obtaining predictive dental arch data 146, e.g., for performance of a corrective action. In some embodiments, features may include segmentation data of dental arch data 142. Segmentation may be performed (e.g., using a trained machine learning model trained to perform instance segmentation or semantic segmentation), and may include separation of oral structure data into various groups of teeth, individual teeth, tissues, portions of tissues, etc. For example, a first segment may include one or more teeth, a second segment may include a palate, a third segment may include an upper or lower gingiva, etc. In a further example, a separate segment may be generated for each individual tooth, which may be identified and labeled in some embodiments (e.g., based on tooth number).

Each instance (e.g., set) of dental arch data 142 may correspond to an individual (e.g., patient), a group of similar dental arches, or the like. Data from a jaw pair or dental arch may be segmented in embodiments. For example, data from a single tooth or group of teeth of a jaw pair or dental arch may be identified, may be separated from data for other teeth, and/or may be stored, along with data of the complete jaw pair or dental arch. The data store may further store information associating sets of different data types, e.g., information indicative that a tooth belongs to a certain jaw pair or dental arch, that a sparse three-dimensional intraoral scan belongs to the same jaw pair or dental arch as a two-dimensional image, or the like.

Predictive dental arch data 146 may share one or more features with historical dental arch data 144. For example, predictive dental arch data 146 may resemble data generated by one or more types of jaw measurement equipment. In some embodiments, predictive system 110 may generate predictive dental arch data 146 using supervised machine learning (e.g., predictive dental arch data 146 includes output from a machine learning model that was trained using labeled data, such as incomplete jaw pair data (e.g., data of one or a few teeth) labeled with complete jaw pair data (e.g., jaw pair data including all teeth of the jaw). In some embodiments, predictive system 110 may generate predictive dental arch data 146 using unsupervised machine learning (e.g., predictive dental arch data 146 includes output from a machine learning model that was trained using unlabeled data, output may include clustering results, principle component analysis, anomaly detection, etc.). In some embodiments, predictive system 110 may generate predictive dental arch data 146 using semi-supervised learning (e.g., training data may include a mix of labeled and unlabeled data, etc.). In some embodiments, predictive system 110 may generate predictive dental arch data 146 using self-supervised learning, e.g., training data may also include target output data, such as in an autoencoder model.

Client device 120, dental arch data capturing equipment 126, predictive server 112, data store 140, server machine 170, and server machine 180 may be coupled to each other via network 130 for generating predictive dental arch data 146, e.g., to generate synthetic versions of dental arch data, to perform corrective actions associated with dental arches, etc. In some embodiments, network 130 may provide access to cloud-based services. Operations performed by client device 120, predictive system 110, data store 140, etc., may be performed by virtual cloud-based devices.

In some embodiments, network 130 is a public network that provides client device 120 with access to the predictive server 112, data store 140, and other publicly available computing devices. In some embodiments, network 130 is a private network that provides client device 120 access to dental arch data capturing equipment 126, data store 140, and other privately available computing devices. Network 130 may include one or more Wide Area Networks (WANs), Local Area Networks (LANs), wired networks (e.g., Ethernet network), wireless networks (e.g., an 802.11 network or a Wi-Fi network), cellular networks (e.g., a Long Term Evolution (LTE) network), routers, hubs, switches, server computers, cloud computing networks, and/or a combination thereof.

Client device 120 may include computing devices such as Personal Computers (PCs), laptops, mobile phones, smart phones, tablet computers, netbook computers, network connected televisions (“smart TV”), network-connected media players (e.g., Blu-ray player), a set-top-box, Over-the-Top (OTT) streaming devices, operator boxes, etc. Client device 120 may include a corrective action component 122. Corrective action component 122 may receive user input (e.g., via a Graphical User Interface (GUI) displayed via the client device 120) of an indication associated with dental arch data 142. In some embodiments, corrective action component 122 transmits the indication to the predictive system 110, receives output (e.g., predictive dental arch data 146) from the predictive system 110, determines a corrective action based on the output, and causes the corrective action to be implemented. In some embodiments, corrective action component 122 obtains dental arch data 142 (e.g., historical dental arch data 144 and/or predictive dental arch data 146) and provides the data to a user via data display component 124.

In some embodiments, corrective action component 122 receives an indication of a corrective action from the predictive system 110 and causes the corrective action to be implemented. Each client device 120 may include an operating system that allows users to one or more of generate, view, or edit data (e.g., historical dental arch data 144, predictive dental arch data 146, etc.).

Corrective actions may be associated with design of a treatment plan, updating of a treatment plan, providing an alert associated with a treatment plan to a user, or the like.

Predictive server 112, server machine 170, and server machine 180 may each include one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc. Operations of predictive server 112, server machine 170, server machine 180, data store 140, etc., may be performed by a cloud computing service, cloud data storage service, etc.

Predictive server 112 may include a predictive component 114. In some embodiments, the predictive component 114 may receive dental arch data 142, (e.g., receive from the client device 120, retrieve from the data store 140) and generate output (e.g., predictive dental arch data 146) based on the input data. In some embodiments, dental arch data 146 may include one or more predicted dimension measurements of one or more dental arches of a jaw pair. In some embodiments, predictive component 114 may use one or more trained machine learning models 190 to determine the output for performing the corrective action based on current data.

System 100 may include one or more machine leaning models, e.g., model 190. Machine learning models may perform many tasks, including mapping dental arch data (e.g., including tooth, gingiva, palate, etc., data) to a latent space, mapping latent space data to dental arch data (e.g., output may include one or more teeth, arches, jaws, tissues, etc.), performing mappings between positions within a latent space (e.g., based on mapping data 162), performing mapping between latent spaces (e.g., based on mapping data 162), or the like. Model 190 may be trained using dental arch data 142. Model 190 may be trained using historical dental arch data 144. Model 190, once trained, may be provided with current dental arch data 143 as input for performing one or more operations, generating predictive dental arch data 146, or the like.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs).

A recurrent neural network (RNN) is another type of machine learning model. A recurrent neural network model is designed to interpret a series of inputs where inputs are intrinsically related to one another, e.g., time trace data, sequential data, etc. Output of a perceptron of an RNN is fed back into the perceptron as input, to generate the next output.

A graph convolutional network (GCN) is a type of machine learning model that is designed to operate on graph-structured data. Graph data includes nodes and edges connecting various nodes. GCNs extend CNNs to be applicable to graph-structured data which captures relationships between various data points. GCNs may be particularly applicable to meshes, such as three-dimensional data.

Many other types and varieties of machine learning models may be utilized for one or more embodiments of the present disclosure. Further types of machine learning models that may be utilized for one or more aspects include transformer-based architectures, generative adversarial networks, volumetric CNNs, etc. Selection of a specific type of machine learning model may be performed responsive to an intended input and/or output data, such as selecting a model adapted to three-dimensional data to perform operations on three-dimensional models of dental arches, a model adapted to two-dimensional image data to perform operations based on images of a patient's teeth, etc.

Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

In some embodiments, predictive component 114 receives current dental arch data 143, performs signal processing to break down the current data into sets of current data, provides the sets of current data as input to a trained model 190, and obtains outputs indicative of predictive dental arch data 146 from the trained model 190.

In some embodiments, the various models discussed in connection with model 190 (e.g., supervised machine learning model, unsupervised machine learning model, etc.) may be combined in one model (e.g., a hierarchical model), or may be separate models.

Data may be passed back and forth between several distinct models included in model 190 and predictive component 114. In some embodiments, some or all of these operations may instead be performed by a different device, e.g., client device 120, server machine 170, server machine 180, etc. It will be understood by one of ordinary skill in the art that variations in data flow, which components perform which processes, which models are provided with which data, and the like are within the scope of this disclosure.

Data store 140 may be a memory (e.g., random access memory), a drive (e.g., a hard drive, a flash drive), a database system, a cloud-accessible memory system, or another type of component or device capable of storing data. Data store 140 may include multiple storage components (e.g., multiple drives or multiple databases) that may span multiple computing devices (e.g., multiple server computers). The data store 140 may store dental arch data 142, and mapping data 162.

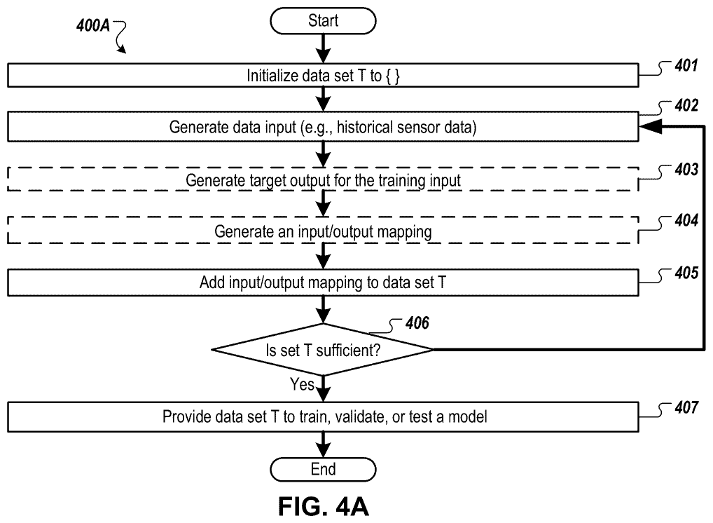

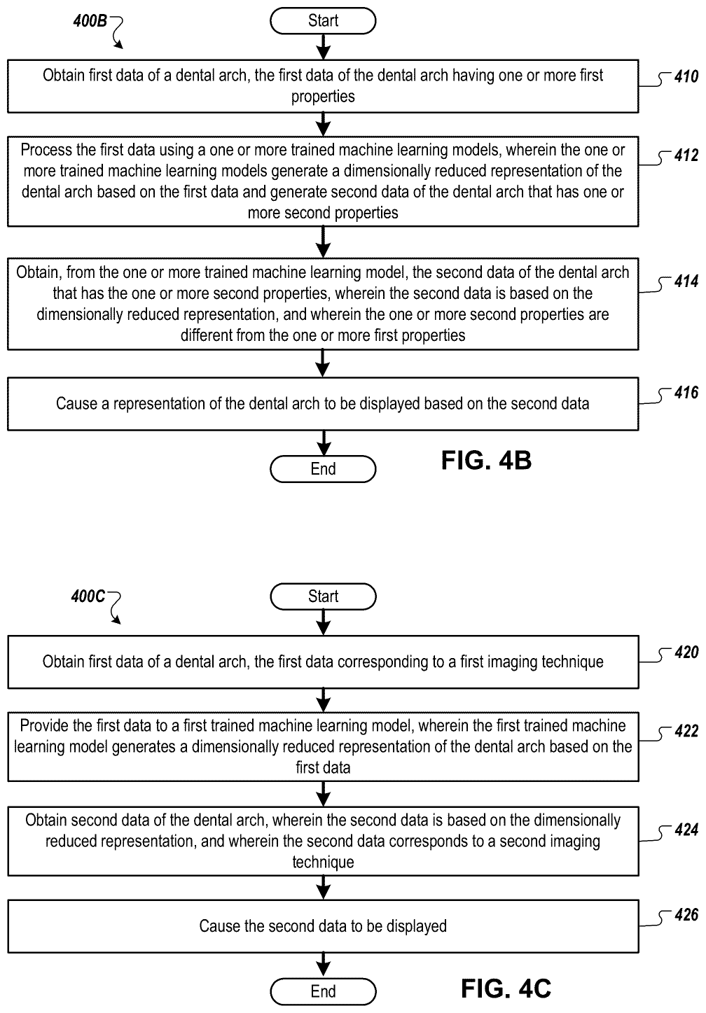

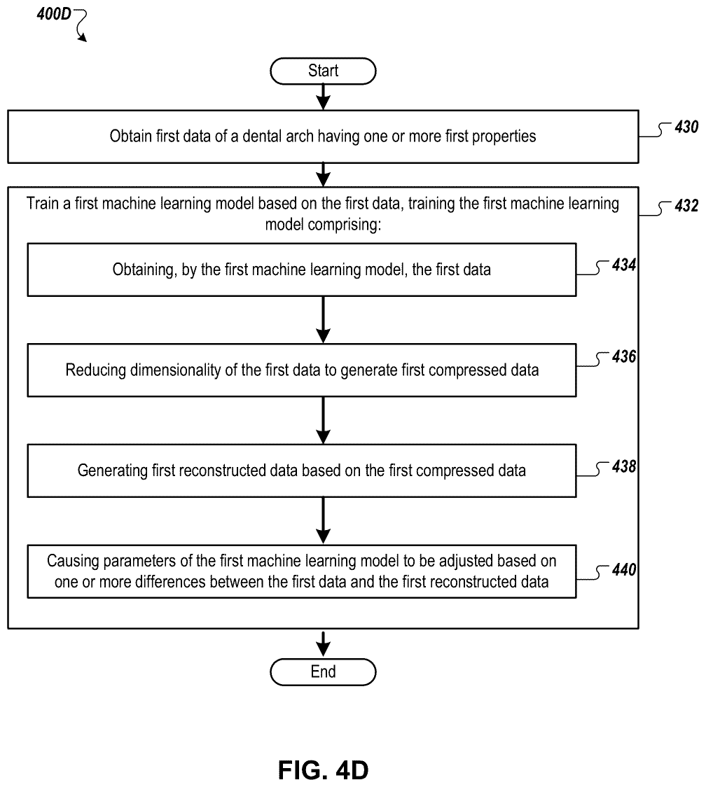

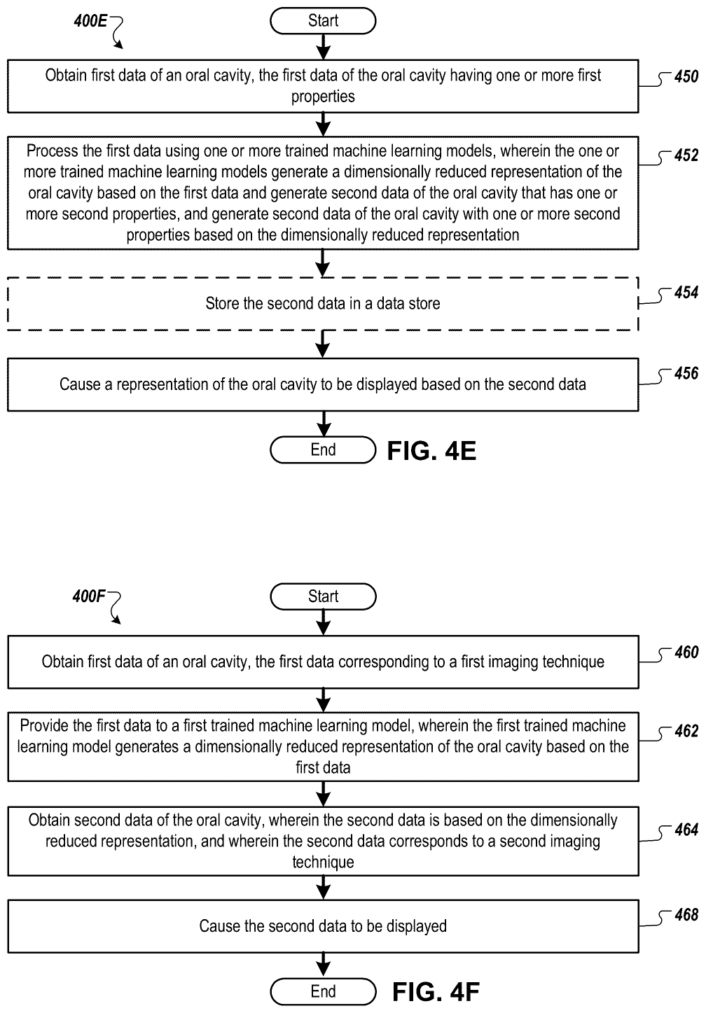

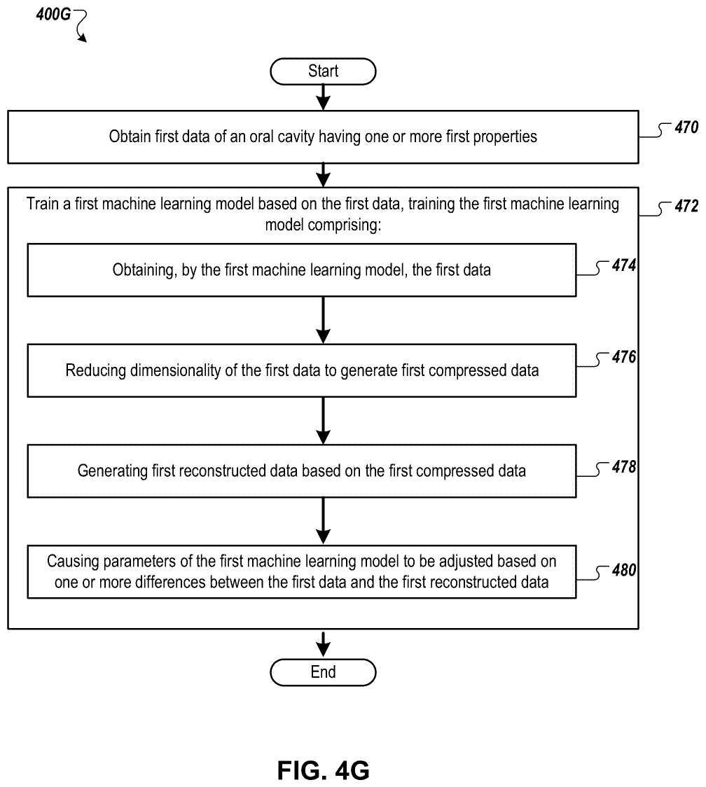

In some embodiments, predictive system 110 further includes server machine 170 and server machine 180. Server machine 170 includes a data set generator 172 that is capable of generating data sets (e.g., a set of data inputs and a set of target outputs) to train, validate, and/or test model(s) 190, including one or more machine learning models. Some operations of data set generator 172 are described in detail below with respect to FIG. 4A. In some embodiments, data set generator 172 may partition the historical data (e.g., historical dental arch data 144) into a training set (e.g., sixty percent of the historical data), a validating set (e.g., twenty percent of the historical data), and a testing set (e.g., twenty percent of the historical data).

In some embodiments, predictive system 110 (e.g., via predictive component 114) generates multiple sets of features. For example a first set of features may correspond to a first subset of dental arch data (e.g., from a first set of teeth, first combination of teeth, first arch of a jaw pair, first combination of some teeth and some other tissues, set of gingiva and/or palate, or the like) that correspond to each of the data sets (e.g., training set, validation set, and testing set) and a second set of features may correspond to a second subset of dental arch data that correspond to each of the data sets.

In some embodiments, machine learning model 190 is provided historical data as training data. The type of data provided will vary depending on the intended use of the machine learning model. For example, a machine learning model may be trained by providing the model with historical dental arch data 144 as training input. The machine learning model 190 may be configured to dimensionally reduce the input data, e.g., the machine learning model 190 may be configured to map the input data to a latent space representing dental arch data (e.g., potentially including associated tissue data, in some embodiments). A machine learning model may be provided with latent space representations of a dental arch (or one or more portions or teeth and/or other oral structures associated with a dental arch) before and after treatment as training input and target output, respectively. Such a machine learning model may be configured to generate mappings (in a latent space) between representations of treated and untreated dental arches (e.g., to predict outcomes of orthodontic treatment).

In one embodiment, server machine 180 includes a training engine 182, a validation engine 184, selection engine 185, and/or a testing engine 186. An engine (e.g., training engine 182, a validation engine 184, selection engine 185, and a testing engine 186) may refer to hardware (e.g., circuitry, dedicated logic, programmable logic, microcode, processing device, etc.), software (such as instructions run on a processing device, a general purpose computer system, or a dedicated machine), firmware, microcode, or a combination thereof. The training engine 182 may be capable of training a model 190 using one or more sets of features associated with the training set from data set generator 172. The training engine 182 may generate multiple trained models 190, where each trained model 190 corresponds to a distinct set of features of the training set (e.g., sensor data from a distinct set of sensors). For example, a first trained model may have been trained using all features (e.g., X1-X5), a second trained model may have been trained using a first subset of the features (e.g., X1, X2, X4), and a third trained model may have been trained using a second subset of the features (e.g., X1, X3, X4, and X5) that may partially overlap the first subset of features. Data set generator 172 may receive the output of a trained model (e.g., a latent space representation of jaw pair data), collect that data into training, validation, and testing data sets, and use the data sets to train a second model (e.g., a machine learning model configured to output predictive data by mapping within and/or between latent spaces representing dental arches, etc.).

Validation engine 184 may be capable of validating a trained model 190 using a corresponding set of features of the validation set from data set generator 172. For example, a first trained machine learning model 190 that was trained using a first set of features of the training set may be validated using the first set of features of the validation set. The validation engine 184 may determine an accuracy of each of the trained models 190 based on the corresponding sets of features of the validation set. Validation engine 184 may discard trained models 190 that have an accuracy that does not meet a threshold accuracy. In some embodiments, selection engine 185 may be capable of selecting one or more trained models 190 that have an accuracy that meets a threshold accuracy. In some embodiments, selection engine 185 may be capable of selecting the trained model 190 that has the highest accuracy of the trained models 190.

Testing engine 186 may be capable of testing a trained model 190 using a corresponding set of features of a testing set from data set generator 172. For example, a first trained machine learning model 190 that was trained using a first set of features of the training set may be tested using the first set of features of the testing set. Testing engine 186 may determine a trained model 190 that has the highest accuracy of all of the trained models based on the testing sets.

In the case of a machine learning model, model 190 may refer to the model artifact that is created by training engine 182 using a training set that includes data inputs and corresponding target outputs (correct answers for respective training inputs). Patterns in the data sets can be found that map the data input to the target output (the correct answer), and machine learning model 190 is provided mappings that capture these patterns. The machine learning model 190 may use one or more of Support Vector Machine (SVM), Radial Basis Function (RBF), clustering, supervised machine learning, semi-supervised machine learning, unsupervised machine learning, k-Nearest Neighbor algorithm (k-NN), linear regression, random forest, neural network (e.g., artificial neural network, recurrent neural network, CNN, graph neural network, GCN), etc. In some embodiments, model 190 may be or comprise an autoencoder. An autoencoder may be configured to reduce dimensionality of input data (e.g., map the input data to a latent space), and then expand the compressed data. An error metric may be minimized during training related to differences between the input and the reconstructed output, e.g., the autoencoder may determine a latent space that accurately maps to dental arches (e.g., including associated gingiva and/or palates or other structures of the oral cavity, in some embodiments).

Predictive component 114 may provide current data to model 190 and may run model 190 on the input to obtain one or more outputs. For example, predictive component 114 may provide current dental arch data 143 to model 190 and may run model 190 on the input to obtain one or more outputs. Predictive component 114 may be capable of determining (e.g., extracting) predictive dental arch data 146 from the output of model 190. Predictive component 114 may determine (e.g., extract) confidence data from the output that indicates a level of confidence that predictive data (e.g., predictive dental arch data 146) is an accurate predictor of dental arch data associated with the input data for dental arches. Predictive component 114 or corrective action component 122 may use the confidence data to decide whether to cause a corrective action associated with the dental arch.

The confidence data may include or indicate a level of confidence that the predictive dental arch data 146 is an accurate prediction associated with the input data. In one example, the level of confidence is a real number between 0 and 1 inclusive, where 0 indicates no confidence that the predictive dental arch data 146 is an accurate prediction for the input data and 1 indicates absolute confidence that the predictive dental arch data 146 accurately predicts properties of a dental arch associated with the input data. Responsive to the confidence data indicating a level of confidence below a threshold level for a predetermined number of instances (e.g., percentage of instances, frequency of instances, total number of instances, etc.) predictive component 114 may cause trained model 190 to be re-trained (e.g., based on current dental arch data 143, historical dental arch data 144, etc.). In some embodiments, retraining may include generating one or more data sets (e.g., via data set generator 172) utilizing historical data.

For purpose of illustration, rather than limitation, aspects of the disclosure describe the training of one or more machine learning models 190 using historical data and inputting current data into the one or more trained machine learning models to determine predictive jaw pair data 168. In other embodiments, a heuristic model, physics-based model, or rule-based model is used to determine predictive jaw pair data 168 (e.g., without or in addition to using a trained machine learning model. Any of the information described with respect to data inputs to one or more models for manipulating jaw pair data may be monitored or otherwise used in the heuristic, physics-based, or rule-based model.

In some embodiments, the functions of client device 120, predictive server 112, server machine 170, and server machine 180 may be provided by a fewer number of machines. For example, in some embodiments server machines 170 and 180 may be integrated into a single machine, while in some other embodiments, server machine 170, server machine 180, and predictive server 112 may be integrated into a single machine. In some embodiments, client device 120 and predictive server 112 may be integrated into a single machine. In some embodiments, functions of client device 120, predictive server 112, server machine 170, server machine 180, and data store 140 may be performed by a cloud-based service.

In general, functions described in one embodiment as being performed by client device 120, predictive server 112, server machine 170, and server machine 180 can also be performed on predictive server 112 in other embodiments, if appropriate. In addition, the functionality attributed to a particular component can be performed by different or multiple components operating together. For example, in some embodiments, the predictive server 112 may determine a corrective action based on the predictive dental arch data 146. In another example, client device 120 may determine the predictive dental arch data 146 based on output from the trained machine learning model.

In addition, the functions of a particular component can be performed by different or multiple components operating together. One or more of the predictive server 112, server machine 170, or server machine 180 may be accessed as a service provided to other systems or devices through appropriate application programming interfaces (API).

In embodiments, a “user” may be represented as a single individual. However, other embodiments of the disclosure encompass a “user” being an entity controlled by a plurality of users and/or an automated source. For example, a set of individual users federated as a group of administrators may be considered a “user.”

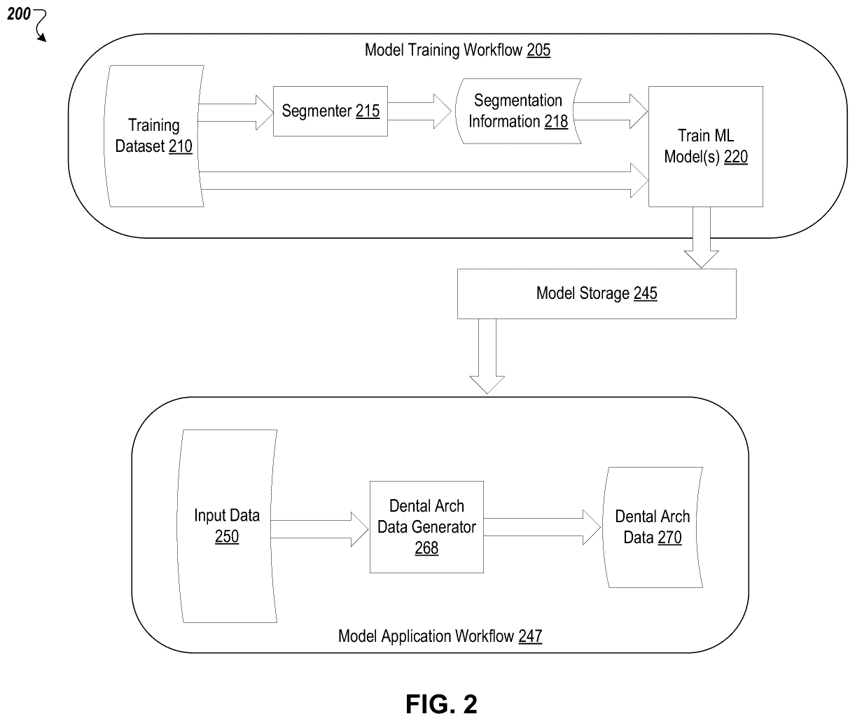

FIG. 2 illustrates workflows 200 for training and implementing one or more machine learning models for performing operations associated with one or more latent spaces representing dental arches (e.g., including any oral structures associated with dentistry, including gingiva, palate, or other structures of the oral cavity), in accordance with embodiments of the present invention. The illustrated workflows include a model training workflow 205 and a model application workflow 247. The model training workflow 205 is to train one or more machine learning models (e.g., deep learning models, generative models, etc.) to perform one or more data segmentation tasks and/or data generation tasks (e.g., for images of smiling persons showing their teeth, intraoral images, images of an impression taken of a dental arch, images of physical or digital three-dimensional dental models, etc.). The model application workflow 247 is to apply the one or more trained machine learning models to generate predictive dental arch data (e.g., jaw pair data) and/or latent space representations of dental arches based on the input data 250.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

The model training workflow 205 and the model application workflow 247 may be performed by processing logic, executed by a processor of a computing device. Workflows 205 and 247 may be implemented, for example, by one or more devices depicted in FIG. 1, such as server machine 170, server machine 180, predictive server 112, etc. These methods and/or operations may be implemented by one or more machine learning modules executed on processing devices of devices depicted in FIG. 1.

For the model training workflow 205, a training dataset 210 containing hundreds, thousands, tens of thousands, hundreds of thousands or more examples of input data may be provided. The properties of the input data will correspond to the intended use of the machine learning model(s). For example, a machine learning model for mapping jaw pair data to a latent space may be trained. Training the machine learning model for mapping jaw pair data to a latent space may include providing a training dataset 210 of jaw pair data to be mapped to the latent space. Training dataset 210 may include various types of data, e.g., various representations of dental arches. Training dataset 210 may include three-dimensional scan data for a machine learning model configured to map three-dimensional scan data to a latent space. Training dataset 210 may include two-dimensional images including jaw pair information for training a machine learning model to map two-dimensional image data to a latent space. Training dataset 210 may include computed tomography (e.g., cone-beam computed tomography), x-ray, or other types of data for training a machine learning model to map the corresponding data type to a latent space. Training dataset 210 may include additional information, such as contextual information, metadata, etc. Training dataset 210 may include positional data associated with one or more teeth, orientation data, spatial relationship data, etc. Training dataset 210 may include data that may be associated with predictions of any type of dental issue that may be expressed in a latent space representing dental arches and/or jaw pairs. For example, results of orthodontic, restorative, or ortho-restorative treatments may be predicted, ongoing tooth wear, tooth decay, changes to teeth due to jaw development (e.g., as a child or adolescent grows), etc.

Training dataset 210 may reflect the intended use of the machine learning model. A model may be configured to predict a change to a dental arch or jaw pair. For example, a model may be configured to predict the result of an orthodontic treatment. The machine learning model configured to predict results of an orthodontic treatment may be provided with data indicative of one or more dental arches before and after orthodontic treatment as part of training dataset 210. The machine learning model may be provided with latent space representations of dental arches before and after treatment and/or with images, intraoral scans, 3D models, etc. of dental arches before and after treatment. The machine learning model may be provided with latent space vectors representing dental arches before and after treatment as training input and target output. Such a model may be trained to receive a latent space representation and map the latent space representation to a new representation (e.g., a new vector, a new location, etc.) of the same latent space. In some embodiments, a first machine learning model is trained to map dental arch data to a point in a latent space and a second machine learning model is trained to map from a point in the latent space to dental arch data in parallel.

As a further example, a model may be configured to predict the results of one stage of treatment to a dental arch or jaw pair. Training dataset 210 may include data indicative of a dental arch before and after a treatment stage as training input and target output, respectively. Training dataset 210 may include data indicative of dental arches represented in a latent space, e.g., latent space vectors.

As a further example, a model may be configured to predict a mapping between latent spaces representing dental arches. For example, a first latent space may be associated with a first jaw pair measurement technique and a second latent space may be associated with a second jaw pair measurement technique, and a machine learning model configured to map from the first latent space to the second latent space may be provided latent space representations of dental arches provided by both measurement techniques as training input and target output.

As a further example, a model may be configured to predict results of allowing a dental issue to progress. For example, continued tooth wear may be predicted by training a model based on dental data before and after a period of time where tooth wear occurred. Untreated tooth decay may be predicted by training a model based on dental data of untreated tooth decay. Changes to a jaw pair, dental arch, one or more teeth, oral tissues, or the like based on a changing or developing jaw (e.g., in a child) may be predicted based on providing training data including data collected from growing or developing patients. As an example related to predicting the course of a dental issue, training dataset 210 may include measured dental arch data from a first patient at a first time, and measured dental arch data from the same patient at a second time. The data from the first time may be projected into the latent space and the data from the second time may be projected into the latent space. A machine learning model may be trained to make predictions of other patients based on progression of tooth wear or other dental issues. A machine learning model may be trained to receiving the latent space data from the first time and the latent space data from the second time and generate predictive latent space data at a third time indicative of a prediction of further development of the dental issue, which may be based on the difference in the latent space between the representation of the jaw pair and the first and second times.

As a further example, training dataset 210 may include data for configuring a machine learning model to predict development of teeth of a patient as the patient grows, as the jaw develops, or the like. Training dataset 210 may include latent space representations of dental arches as patient's jaw develop, e.g., through adolescence. One or more machine learning models may be trained to predict changes to teeth of a patient as the patient ages. One or more machine learning models may be trained to predict changes to teeth of a patient if a developing dental issue is not treated. One or more machine learning models may be trained to predict changes to developing teeth of a patient if a developing dental issue is treated. Predictions with and without treatment may be compared to enable a patient and dental practitioner to make treatment determinations.

In some embodiments, some or all of the training dataset 210 may be segmented. For example, a model may be trained to map incomplete or sparse jaw pair data to a latent space representing dental arches. The segmenter 215 may separate portions of dental arch data for training of a machine learning model. For example, individual teeth of dental arch data may be utilized as training input for a model configured to map data of one or more teeth to a latent space representing dental arches. Individual teeth, groups or sets of teeth, arches, jaws, oral tissues, portions of oral tissues, combinations of these (e.g., a subset of teeth with their associated regions of gingiva) or the like may be segmented from jaw pair or dental arch data to train a model to complete incomplete jaw pair data, to map incomplete dental arch data or jaw pair data to a latent space representing jaw pairs, to map incomplete dental arch data to a latent space representing complete dental arches, or the like. For example, dental arch data of just an upper or lower dental arch may be received and processed by one or more trained machine learning models, and the one or more trained machine learning models may output dental arch data for a jaw pair including both the upper and lower dental arch.

Data of the training dataset 210 may be processed by segmenter 215 that segments the data of training dataset 210 (e.g., jaw pair data) into multiple different features. The segmenter may then output segmentation information 218. The segmenter 215 may itself be a machine learning model, e.g., a machine learning model configured to identify individual teeth or target groups of teeth from dental arch data. Segmenter 215 may perform image processing and/or computer vision techniques or operations to extract segmentation information 218 from data of training dataset 210. In some embodiments, segmenter 215 may not include a machine learning model. In some embodiments, training dataset 210 may not be provided to segmenter 215, e.g., training dataset 210 may be provided to train ML models without segmentation.

In some embodiments, various other pre-processing operations (e.g., in addition to or instead of segmentation) may also be performed before providing input (e.g., training input or inference input) to the machine learning model. Other pre-processing operations may share one or more features with segmenter 215 and/or segmentation information 218, e.g., location in the model training workflow 205. Pre-processing operations may include mesh closing, artifact removal, registration (bringing various meshes into the same topology), or other pre-processing that may improve performance of the machine learning models.

Data from training dataset 210 may be provided to train one or more machine learning models at block 220. Training a machine learning model may include first initializing the machine learning model. The machine learning model that is initialized may be a deep learning model such as an artificial neural network. An optimization algorithm, such as back propagation and gradient descent may be utilized in determining parameters of the machine learning model based on processing of data from training dataset 210.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many inputs in the training dataset yields a network that can produce correct output when presented with inputs that are different than the ones present in the training dataset. In high-dimensional settings, such as large images, this generalization is achieved when a sufficiently large and diverse training dataset is made available.

An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer.

Processing logic adjusts weights of one or more nodes in the machine learning model(s) based on an error term. The error term may be based upon a difference between output of the machine learning model and target output provided as part of training dataset 210. The error term may be based on a difference between output of the machine learning model and training input, such as in the case of an autoencoder configured to reduce dimensionality of input data (e.g., map the data to a latent space) and then reconstruct the input data based on the compressed (dimensionally reduced) data. In some embodiments, the error term may be based on the output of a discriminator model. The discriminator model may be a machine learning model, a rule-based model, a heuristic model, or the like, that is configured to classify or determine a quality of the output of a machine learning model. The parameters of the machine learning model being trained may be adjusted based on the output of the discriminator model(s). The output of the discriminator model(s) may be used to determine an error term or delta for each node in the generator model(s) and/or discriminator model(s). Based on this error, the artificial neural networks adjust one or more of their parameters for one or more of their nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

In some embodiments, portions of available training data (e.g., training dataset 210) may be utilized for different operations associated with generating a usable machine learning model. Portions of training dataset 210 may be separated for performing different operations associated with generating a trained machine learning model. Portions of training dataset 210 may be separated for use in training, validating, and testing of machine learning models. For example, 60% of training dataset 210 may be utilized for training, 20% may be utilized for validating, and 20% may be utilized for testing.

In some embodiments, the machine learning model may be trained based on the training portion of training dataset 210. Training the machine learning model may include determining values of one or more parameters as described above to enable a desired output related to an input provided to the model. One or more machine learning models may be trained, e.g., based on different portions of the training data. The machine learning models may then be validated, using the validating portion of the training dataset 210. Validation may include providing data of the validation set to the trained machine learning models and determining an accuracy of the models based on the validation set. Machine learning models that do not meet a target accuracy may be discarded. In some embodiments, only one machine learning model with the highest validation accuracy may be retained, or a target number of machine learning models may be retained. Machine learning models retained through validation may further be tested using the testing portion of training dataset 210. Machine learning models that provide a target level of accuracy in training operations may be retained and utilized for future operations. At any point (e.g., validation, testing), if the number of models that satisfy a target accuracy condition does not satisfy a target number of models, training may be performed again to generate more models for validation and testing.

Once one or more trained machine learning models are generated, they may be stored in model storage 245, and utilized for generating predictive data associated with dental arches, such as providing predictions of one or more missing or partially missing teeth of a jaw pair, providing predictions of a jaw pair based on sparse scan data, providing predictions of results of a measurement technique on jaw pair data based on other measurement technique data, providing predictions of results of a treatment plan or one or more stages of a treatment plan, providing predictions of how treatment will shift or affect oral tissues, or the like. In some embodiments, some oral structures may be treated differently, e.g., a model may be configured to maintain the shape of teeth rigidly, but be configured to allow tissues to shape themselves differently as stages of treatment progress.

In some embodiments, model application workflow 247 includes utilizing the one or more machine learning models trained at block 220. Machine learning models may be implemented as separate machine learning models or a single combined (e.g., hierarchical) machine learning model in embodiments.

Processing logic that applies model application workflow 247 may further execute a user interface, such as a graphical user interface. A user may select one or more options using the user interface. Options may include selecting which of the trained machine learning models to use, selecting which of the operations the trained machine learning models are configured to perform to execute, customizing input and/or output of the machine learning models, or the like. For example, a user may only be interested in output of a subsection of a jaw pair (e.g., only front teeth for a smile reconstruction, only an upper jaw for treatment of an upper jaw malocclusion, etc.) and may request only output related to the target subsection. The user interface may additionally provide options that enable a user to select values of one or more properties. For example, a training plan which focuses on particular movements of target teeth may be selected. The user interface may additionally provide options for selecting an input imaging modality (e.g., first type of dental arch data such as intraoral scans, 3D models, 2D images, etc.) and/or an output imaging modality (e.g., a second type of dental arch data).

Input data 250 is provided to a machine learning model trained in block 220. The input data 250 may correspond to at least a portion or training dataset 210, e.g., be the same type of data, data collected by the same measurement technique, data that resembles data of training dataset 210, or the like. Input data 250 may include dental arch data, dental arch measurement data, dental arch latent space representation data, etc. Input data 250 may further include ancillary information, metadata, labeling data, etc. For example, data indicative of a location, orientation, or identity of a tooth or patient, data indicative of a relationship (e.g., a spatial relationship) between two teeth, a tooth and jaw, two dental arches, or the like, or other data may be included in input data 250 (and training dataset 210).

In some embodiments, input data may be preprocessed. For example, preprocessing operations performed on the training dataset 210 may be repeated for at least a portion of input data 250. Input data 250 may include segmented data, data with anomalies or outliers removed, data with manipulated mesh data, or the like.

Input data is provided to dental arch data generator 268. Dental arch data generator 268 generates dental arch data 270 (e.g., predictive dental arch data 146 of FIG. 1) based on the input data 250. In some embodiments, dental arch data generator 268 includes a single trained machine learning model. In some embodiments, dental arch data generator 268 includes a combination of multiple trained machine learning models. For example, a first trained machine learning model may map input data 250 to a latent space (e.g., may generate a dimensionally reduced representation of the input data 250 that corresponds to a location in the latent space) and a second trained machine learning model may generate synthetic dental arch data 270 from the latent space (e.g., from the dimensionally reduced representation of the input data 250). In some embodiments, dental arch data generator 268 may include a combination of machine learning models and other models. For example, a combination of machine learning models and numerical optimization models may be included in dental arch data generator 268. For example, a projection into a latent space may be performed via numerical optimization, some remapping in the latent space (representing, for example, results of a treatment or a passage of time) may be performed by a machine learning model, and numerical optimization methods may again be used to generate dental arch data 270 based on the remapped latent space data (in some embodiments, including data related to one or more teeth and/or one or more oral tissues). In some embodiments, a corrective action may be performed based on the dental arch data 270. The corrective action may include providing an alert to a user, designing a treatment plan, updating a treatment plan, or the like.

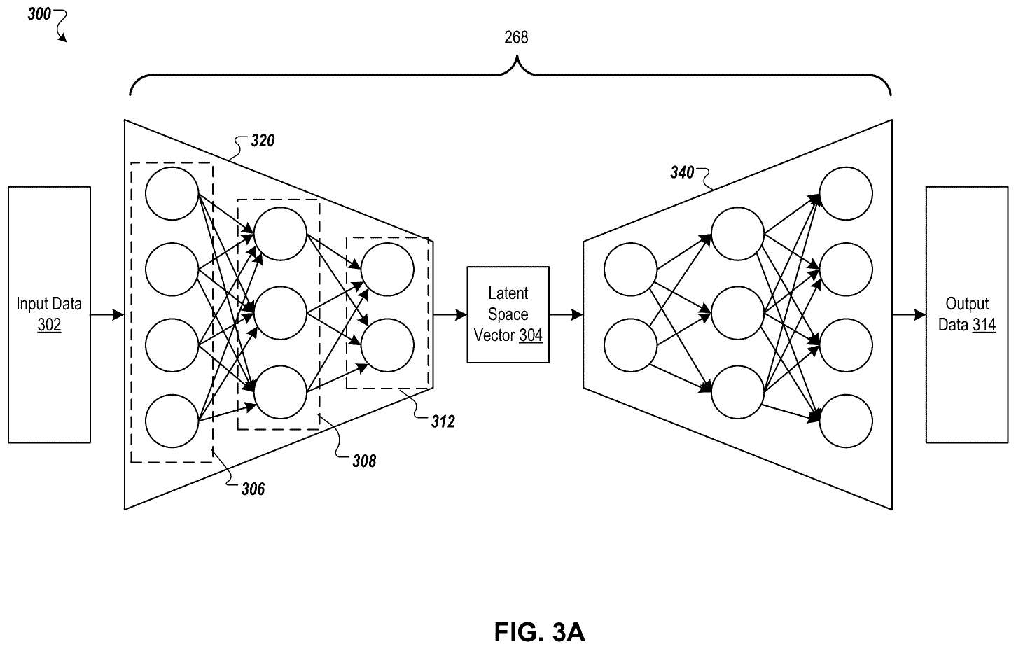

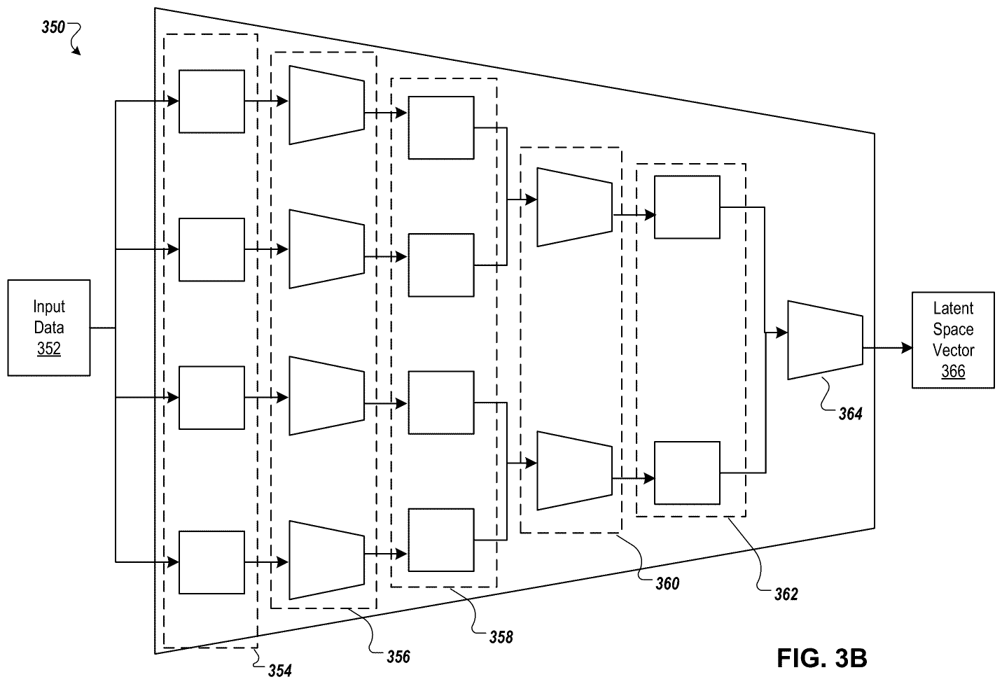

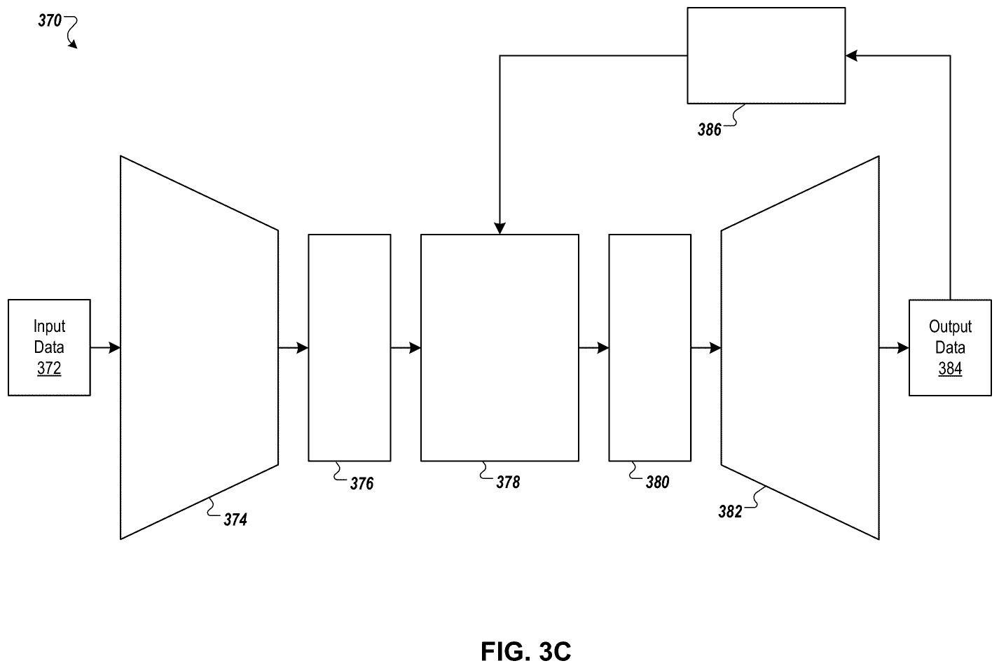

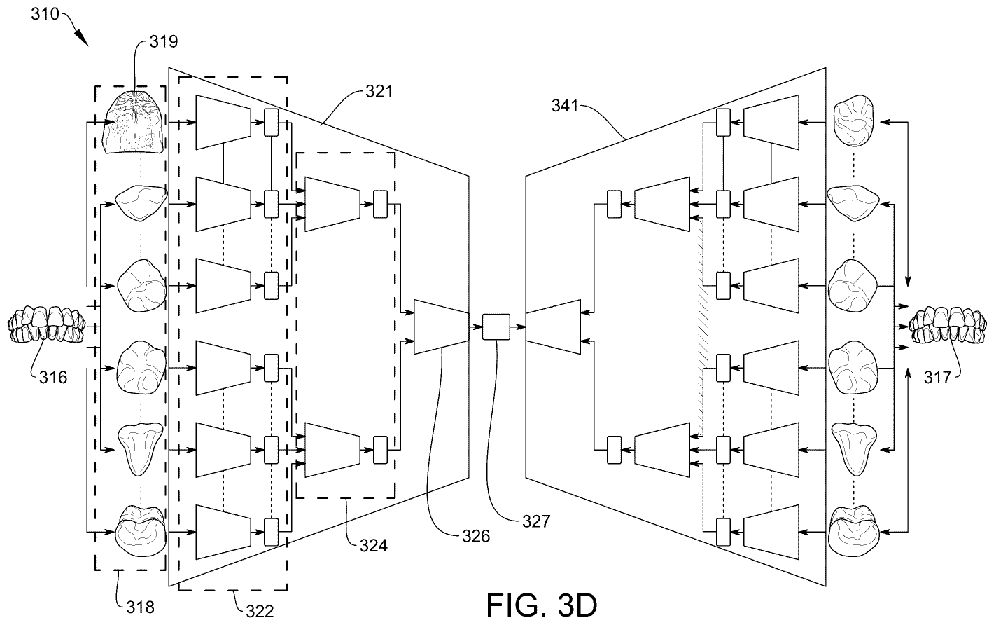

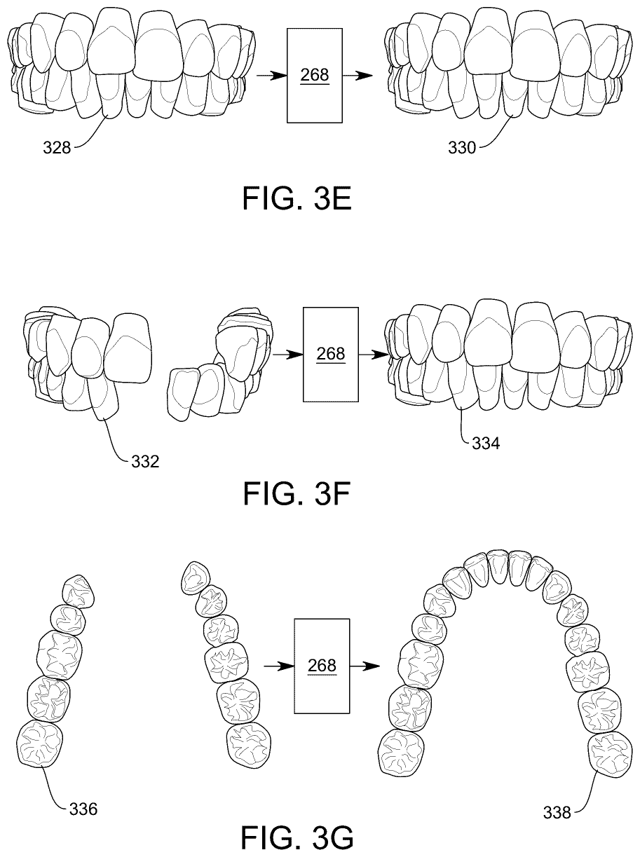

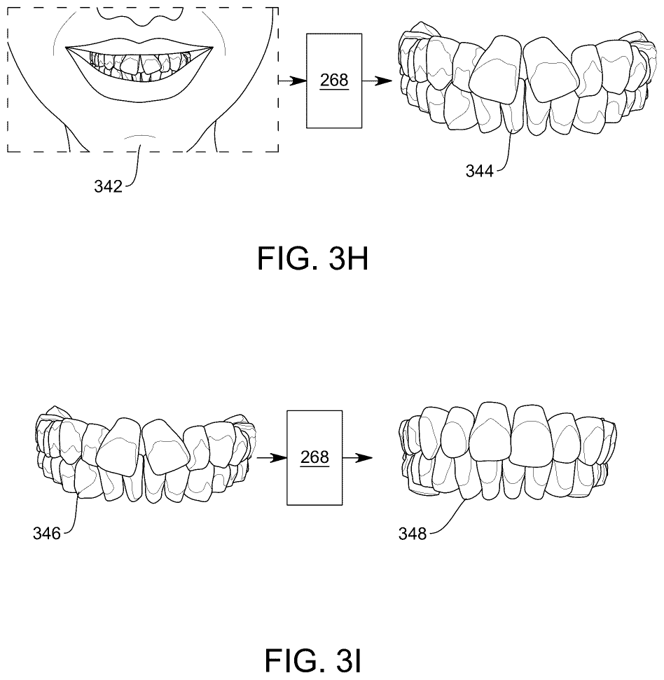

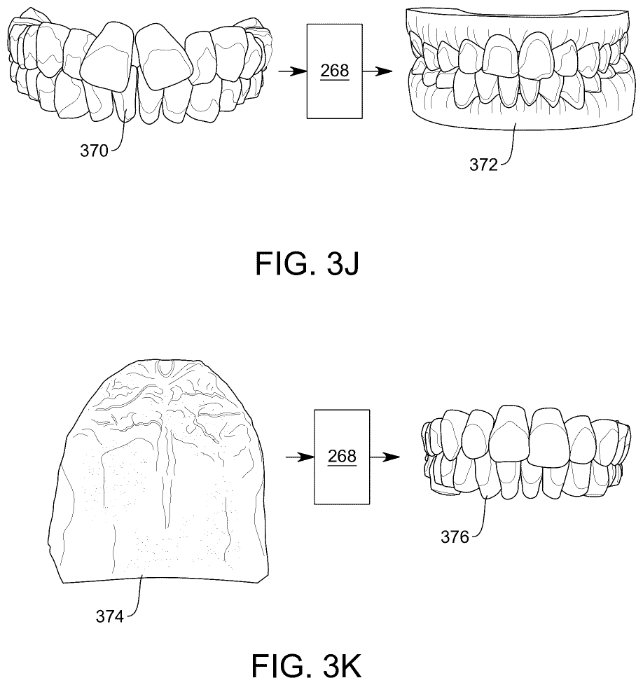

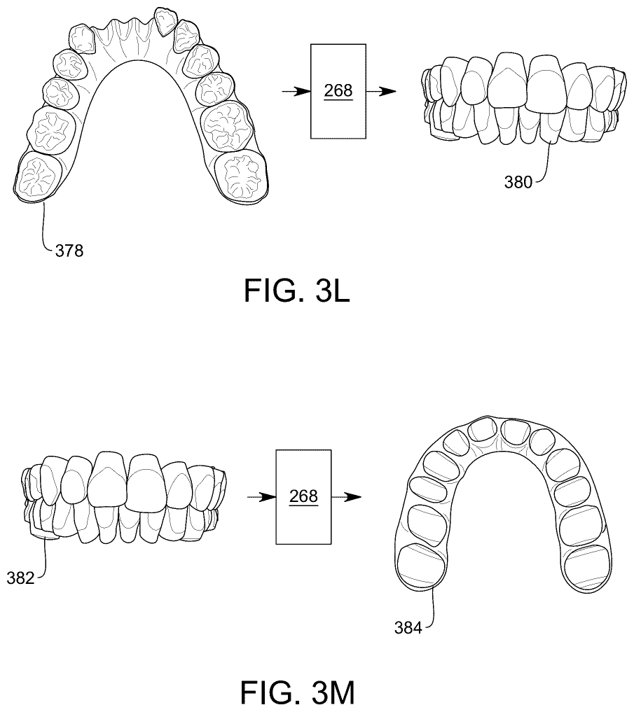

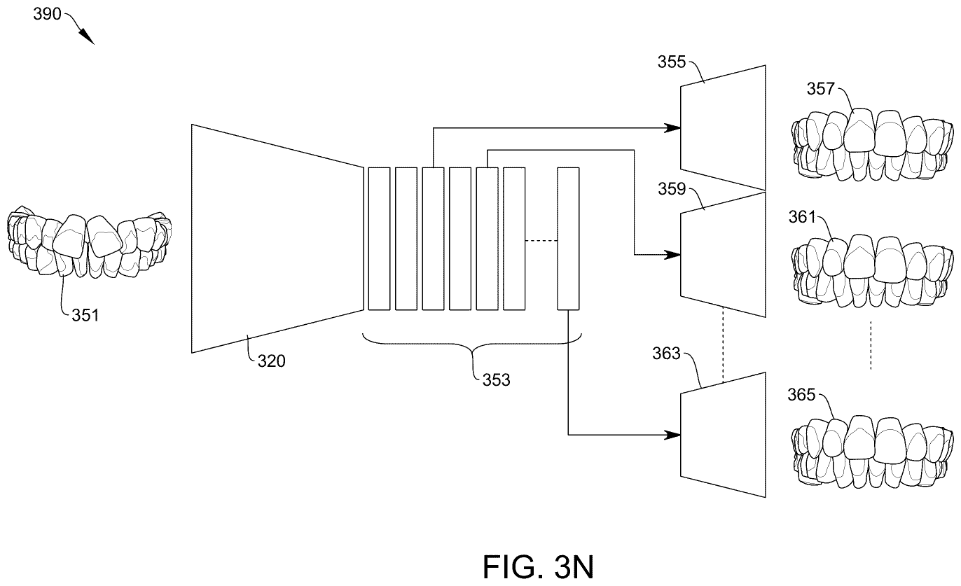

FIG. 3A is a diagram depicting operations of a model 300 (e.g., a machine learning model, an autoencoder model, etc.) that is configured to reduce dimensionality of input data, according to some embodiments. Input data 302 of model 300 may be data of a dental arch or jaw pair, such as three-dimensional scan data, computed tomography data, two-dimensional image data, 3D models of dental arch(es), etc.

The model 300 includes a first portion 320 (e.g., an encoder) and a second portion 340 (e.g., a decoder model). The first portion 320 may dimensionally reduce input data 302 to generate latent space vector 304. The latent space vector 304 may otherwise be referred to as compressed data, a latent space representation of input data 302, a dimensionally reduced representation of input data 302, or the like. During training of the machine learning model 300, the first portion 320 may find functions to fit input data 302 to a compressed latent space vector 304. The compression may take place over several stages (e.g., the first layer 306, second layer 308, and third layer 312 depicted in FIG. 3A). More or fewer stages than depicted may participate in dimensional reduction of input data 302. In some embodiments, data compression may be performed in a single stage.

Second portion 340 receives as input a latent space vector 304 and produces output data 314. Output data 314 may be predictive data (e.g., predictive dental arch data 146 of FIG. 1). Output data 314 may be reconstructed input data or synthetic dental arch data. Output data 314 (and input data 302) may take a variety of forms, including three-dimensional model data, two-dimensional image data, computed tomography or X-ray data, one or more images rendered from three-dimensional data, etc. During training, model 300 may be configured to adjust various parameters (e.g., weights represented by arrows connecting neurons in FIG. 3A) to minimize a difference between input data 302 and output data 314. The minimization function used to train model 300 may also enforce penalties on the dimensionality of the latent space vector 304, e.g., to avoid returning a function with insufficient compression. In some embodiments, different portions of input data 302 may serve as training input and target output. For example, a model may be configured to map two-dimensional image data of teeth of a jaw pair to the latent space vector 304. The latent space vector 304 may be decoded with a decoder associated with three-dimensional jaw pair models to three-dimensional jaw pair predictive data as output data 314. The predictive three-dimensional jaw pair data may be compared to measured or input three-dimensional jaw pair scans to determine an error in the machine learning model encoding.

The function(s) utilized by the first portion 320 and the second portion 340 may be non-linear in nature. In some embodiments, operations may be performed on a latent space vector 304 (e.g., first portion 320 may be the machine learning model that is utilized for a target application). In some embodiments, jaw pair data may be extracted from a latent space for further use (e.g., second portion 340 may be the machine learning model utilized for the target application). In some embodiments, capabilities of both first portion 320 (e.g., encoder) and second portion 340 (e.g., decoder) may be utilized.

For example, a three-dimensional scan may be compressed by mapping the scan data to latent space vector 304, using first portion 320 of model 300. The three-dimensional scan may be used to generate a dimensionally reduced representation of the dental arch or jaw pair. The latent space may be expressive enough to represent many variations of dental arches, jaws, teeth, oral tissues, etc. The latent space may be designed to be sufficiently expressive to represent many variations of dental arches, e.g., via selection of a broad array of dental arches to be included in input data 302. In some embodiments, several models may be utilized to map dental arches to the same latent space. For example, a model may be trained to map three-dimensional intraoral scans (or portions of three-dimensional intraoral scans) to a latent space representing dental arches. A second model may be trained to map one or more two-dimensional images of dental arches (e.g., images of a patient's smile) to the same latent space. A third model may be trained to map another type of input data, such as computed tomography data, to the same latent space. A fourth model may be trained to map a 3D model of a dental arch to the same latent space. In some cases, one or more of these functions may be performed by machine learning models, statistical models, numerical optimization methods, etc. In some embodiments, several latent spaces may be used, e.g., different latent spaces associated with different measurement techniques, different latent spaces associated with dental arches before and after orthodontic treatment, with different malocclusions, with different dental problems or issues, with healthy dental arches, or the like. Other machine learning models may be incorporated into a workflow. For example, a machine learning model may be configured to predict mappings within a latent space, for example mapping from a latent space representation of a dental arch or jaw pair including one or more tooth misalignments or malocclusions to a representation of the dental arch or jaw pair after treatment. As another example, a machine learning model may be configured to map a representation of a dental arch or jaw pair from one latent space to another latent space, for example between latent spaces associated with different dental arch or jaw pair measurement techniques. The progress of any dental problem and any dental correction may be predicted using a latent space representation. For example, progression of tooth wear, tooth decay, patient growth, etc., may be predicted based on latent space representations of jaw pairs, progression or orthodontic, ortho-restorative, or restorative (e.g., replacement or repair of damaged or missing teeth) treatments may be predicted based on latent space representations of jaw pairs, etc. Tooth crowding in a growing patient, e.g., with a narrow dental arch, may be predicted.

Once a latent space is generated (e.g., the latent space in which latent space vector 304 is projected), properties of the latent space may be utilized for dental arch data independent of a particular data input (e.g., input data 302). Data may be sampled from the latent space for generating predictions, performing analysis, or the like. For example, the latent space may be sampled randomly to provide a random assortment of jaw pair representations, or regions that are commonly mapped to by first portion 320 may be sampled to generate similar or likely jaw pair representations. These sampling methods may provide synthetic dental data for other processes, e.g., training of machine learning models that require a large volume of jaw pair data, latent space data, or the like. Further, clusters in the latent space may be identified, e.g., to identify common groupings of properties of input oral data. In some embodiments, sampling may be performed from a cluster, or from a region of the latent space between multiple clusters, to attempt to find regions of a latent space exhibiting some properties of multiple clusters of data, related to some target combination of properties of jaws, teeth, or the like. Latent space representations of dental arches, jaw pairs, teeth, or the like that are similar by one or more similarity metrics (e.g., distance metrics, cosine distance, or the like) may indicate that the associated dental arches are also similar in some way outside the latent space.

For example, data associated with a number of dental arches (e.g., a large number of dental arches) may be provided to first portion 320 to generate a number of latent space vectors. Analysis may be performed on the latent space vectors to determine relationships between locations in the latent space and properties of the dental arches. For example, clustering analysis may be performed on the latent space vectors to determine general positions where latent space jaw representations are likely to be mapped. Clusters may be related to properties of jaws, dental arches, or teeth. For example, a first cluster may correspond to a particular type or location of dental misalignment, a second cluster may correspond to a particular type of tooth or jaw damage, or the like. General jaw pair or dental arch data related to the cluster may be generated, e.g., by second portion 340. Any further processes described herein may be performed based on randomly sampled latent space representations, cluster-sampled latent space representations, or the like. Synthetic jaw pair data, synthetic dental arch data, etc., may be utilized in determining dental issue trends, determining trends in treatment, designing descriptions of typical treatment or issue development, etc.

FIG. 3B is an example machine learning encoder 350 for mapping dental arch data to a latent space, according to some embodiments. Machine learning encoder 350 may be similar to the first portion 320 of machine learning model 300 of FIG. 3A. Encoder 350 is an example, and further embodiments with more or fewer components are within the scope of this disclosure.

Input data 352 is provided to encoder 350. Input data 352 includes dental arch (e.g., including teeth and other oral structures) data. In some embodiments, input data 352 includes three-dimensional scans of dental arches. Input data 352 is segmented into first segmented data 354. The first segmented data 354 may include various categories of data separated from input data 352. In some embodiments, segments of first segmented data 354 may be mutually exclusive. In some embodiments, segments of first segmented data 354 may include overlapping data. Segments of first segmented data 354 may include further data in addition to dental arch data, for example, user selection of one or more parameters. As an example, input data 352 may include intraoral scan data (e.g., a heightmap or a collection of 3D points) of a dental arch or jaw pair, and each segment of first segmented data 354 may include a portion of the intraoral scan data (such as each segment including a scan of a single tooth, each segment including a scan of a group of teeth, some segments including one or more teeth and some segments including at least a portion of one or more oral tissues, or the like).

Segmented data 354 may be provided to a number of modules of first module set 356. Each of the modules of first module set 356 may be a machine learning model. Each of the modules of first module set 356 may be an encoder. Each of the modules of first module set 356 may be configured to reduce dimensionality of input data.

First output data 358 includes output of the modules of first module set 356. In some embodiments, first output data 358 may include reduced dimensional data mapped from each segment of first segmented data 354 based on mapping performed by the corresponding modules of first module set 356. Each segment of first output data 358 may be a latent space representation of the associated data segment.

First output data 358 may be provided to second module set 360. Second module set 360 may share one or more features with first module set 356. Second module set 360 may include multiple encoder models, configured to reduce dimensionality of data input. Second module set 360 may determine mappings to a latent space of groups of categories of segmented data 354. Second module set 360 may determine second output data 362. For example, data associated with groups of teeth, a complete arch, one jaw of a jaw pair, or the like, may be operated on by modules of second module set 360. Second output data 362 may further be provided to third module 364, which may be a final encoder for mapping the data inputs to latent space vector 366.

In some embodiments, a first set of encoders may map individual teeth to latent spaces, a second set of encoders may map full arches to latent spaces, and a third encoder may map a jaw pair to a latent space. In some embodiments, a module of a set of modules may predict a mapping to a latent space based on incomplete data. For example, a jaw pair may be missing one or more teeth, missing portions of one or more teeth (e.g., partial absence of teeth due to teeth being chipped or broken), or the like, and one or more encoders may operate based on the remaining teeth, based on encodings to latent spaces associated with remaining teeth, based on the remaining portions of the broken teeth, or the like. In some embodiments, a decoder may perform similar or identical operations to the encoder, in reverse. For example, a decoder to map a latent space vector to jaw pair data may include a mirroring set of components to encoder 350.

FIG. 3C depicts operations for training or operating a model for mapping data representing dental arches, according to some embodiments. Input data 372 is provided to encoder 374. Encoder 374 may map dental arch data (which may further include any additional oral structures, such as gingiva, palate, etc.) to a latent space representation 376. A mapping model 378 may receive latent space representation 376. Mapping model 378 may be configured to map latent space representation 376 to a different region within the latent space of latent space representation 376. Mapping model 378 may be configured to map latent space representation 376 to a different latent space. Mapping model 378 may generate remapped latent space representation 380 based on latent space representation 376 input. Mapping model 378 may be configured to perform operations on latent space representation 376 related to dental arch transformations. For example, mapping model 378 may map latent space representations of dental arches before orthodontic treatment to latent space representations of dental arches after orthodontic treatment. As another example, mapping model 378 may map latent space representations of dental arches before a stage of treatment to latent space representations of dental arches after a stage of treatment (e.g., orthodontic treatment, restorative treatment, ortho-restorative treatment, etc.). As another example, mapping model 378 may map latent space representations of dental arches as dental conditions progress, such as tooth wear, tooth decay, developing jaw of a growing patient, etc. As another example, mapping model 378 may map latent space representations of dental arches from a latent space associated with one type of jaw pair data (e.g., a type of jaw pair measurement) to a latent space associated with a second type of jaw pair data. As another example, mapping model 378 may map latent space representations of dental arches from a latent space including a first set of data, such as dental arch data, to a different set of data, such as data including oral tissues such as gingiva and/or an upper palate. As another example, mapping model 378 may map data before one or more treatments, including orthodontic treatments, restorative treatments (e.g., crowns or implants), extractions, etc., to data associated with oral condition after the one or more treatments. Mapping model 378 may be a trained machine learning model. Mapping model 378 may be a numerical optimization model.

In some embodiments, different constraints may be imposed on various segments of input data 372. For example, teeth of a dental arch may be constrained in mapping (e.g., via a numerical optimization model) to be strictly rigid, while their relationship to each other is allowed to change. However, shapes of oral tissues, including gingiva, palate, etc., may be allowed to deform to accommodate changes in teeth positioning due to orthodontic treatments, restorative treatments, and/or extractions.

Remapped latent space representation 380 is provided as input to decoder 382. Decoder 382 may predict jaw pair data (e.g., predicted three-dimensional scan data) based on the remapped latent space representation 380. Decoder 382 may generate output data 384 based on the remapped latent space representation 380.

Output data 384 may be provided to discriminator 386. Discriminator 386 determines whether output data 384 is a valid output for input data 372. Discriminator 386 may be a machine learning model, a heuristic or rule-based model, or another module capable of determining whether output data 384 is a valid output associated with input data 372. Discriminator 386 may generate an error term describing how much output data 384 resembles expected output. Output of discriminator 386 may be used to adjust, update, or optimize operations of mapping model 378. Discriminator 386 and mapping model 378 may perform numerical optimization operations. Discriminator 386 may perform operations for training mapping model 378. Discriminator 386 may perform optimization operations during inference of mapping model 378.

In some embodiments, an arrangement including optimization operations may include multiple discriminators. For example, a series of models may each predict results of one stage of jaw pair treatment. Each prediction model (e.g., mapping model such as mapping model 378) may include an associated discriminator configured to determine whether the output of the prediction model is a likely valid output.

FIG. 3D depicts operations of autoencoder 310, according to some embodiments. Autoencoder 310 includes encoder 321 and decoder 341 (e.g., which may share one or more features with first portion 320 and decoder 340 of FIG. 3A). Encoder 321 is provided with input data for processing. Encoder 321 may be provided with input data that is to be dimensionally reduced, projected into a latent space, or the like.

In some embodiments, input data 316 may include data indicative of a patient's teeth. For example, input data 316 may include three-dimensional model data of an upper and/or lower dental arch, as shown in FIG. 3D. In some embodiments, input data 316 may be two-dimensional dental arch data, incomplete or sparse dental arch data, dental arch data including one or more properties (e.g., including misalignment, malocclusion, tooth wear, tooth decay, or the like), etc.

In some embodiments, input data 316 may be segmented into various segmentations 318. As depicted in FIG. 3D, each segment may be a tooth or other structure of the input data 316. As an example, shown in FIG. 3D, segmentations 318 include a number of individual teeth, as well as gingiva 319. Any other combination of oral structures, including only teeth, only tissues, or combinations of teeth and tissues may be used for some applications. In some embodiments, segments may be groups of teeth, separation into an upper and lower dental arch, one or more teeth with associated portions of upper and/or lower, gingiva, palate or other oral structures, or the like. Each portion of segmentations 318 may be provided to an encoder of encoder layer 322. Separate encoders of encoder layer 322 may each provide a latent space vector associated with the corresponding segment provided to the encoder. The encoders may all share one or more parameters, such as weights or biases. Each of the encoder models of encoder layer 322 may be the same. The encoders of encoder layer 322 may be different, for example each may be individually trained on a specific tooth type. The encoders of encoder layer 322 may be configured to determine a latent space representation of the associated segments. The encoder of encoder layer 322 may be configured to predict a latent space representation of an entire jaw pair, dental arch, group of teeth, or the like based on the associated segment.

Latent space representations of encoder layer 322 may be provided to encoders of encoder layer 324. The encoders of encoder layer 324 may be configured to synthesize latent space representations provided by encoders of encoder layer 322 to generate new latent space representations. The new latent space representations may include features indicated by representations of encoder layer 322. In some embodiments, the encoders of encoder layer 324 may group data together in a physical way, such as including a first encoder for an upper dental arch and a second encoder for a lower dental arch. In some embodiments, more or fewer encoder layers may be included in encoder 321. By arranging encoders into a hierarchical encoder model, including other machine learning models, as depicted in FIG. 3D, the encoder may have an advantage in producing useful latent space representations based on incomplete input data. For example, an encoder of encoder layer 324 may receive several latent space representations associated with teeth that are represented in input data 316, and may be capable of predicting properties of teeth not represented in input data 316 based on historical jaw pair data and/or dental arch data provided to the model which includes teeth that are in some way similar to those represented in input data 316. The models of the modeling layers may be utilized by encoder 321 via aggregation, boosting, stacking, blending, or another method of combining outputs of the individual models to generate latent space representation 327.

A final encoder layer 326 may generate a latent space vector representative of a target jaw pair, target dental arch, target group of teeth, target tooth, or the like. The final encoder layer 326 may generate a latent space representation 327, which may be further utilized for any purposes described herein. In some embodiments, one or more transformations may be performed on latent space representation 327, latent space representation 327 may be provided to decoder 341 (which may include reversed operations of encoder 321) to generate output data 317, or the like.

In some embodiments, encoder 321 may include a plurality of encoder models. Each of the encoder models may be configured to make predictions or generate output data based on a segment of jaw pair data. Some of the encoder models included in encoder 321 may be configured to receive data as input indicative of a single tooth, indicative of a specific group of teeth, indicative of a dental arch (e.g., upper or lower arch), or the like.

FIGS. 3E-M depict example operations of a dental arch data generator. FIG. 3E depicts an example operation of dental arch data generator 268 (e.g., dental arch data generator 268 of FIG. 2), according to some embodiments. Input data 328 may be provided to dental arch data generator 268. Dental arch data generator 268 may utilize a dimensionality reduction to generate output data 330. One example of input data as shown in FIG. 3E includes three-dimensional model data of teeth, one or more dental arches, a jaw pair, or the like.

Dental arch data generator 268 may provide output data based on the input data. For example, output data 330 may be a three-dimensional model of one or more teeth, one or more dental arches, a jaw pair, or the like. In one embodiment, input data 328 and output data 330 may be indicative of a jaw pair and/or dental arch with the same properties. For example, in training components of dental arch data generator 268, a comparison may be made between input data 328 and output data 330 to adjust parameters of dental arch data generator 268 for more powerful predictions. In another example, sparse data may be used as input data 328 (e.g., data from an incomplete or low resolution scan of a dental arch) which may be utilized as input to dental arch data generator 268 to produce a more complete and/or denser set of data associated with the same set of teeth as the sparse input data.

FIG. 3F depicts example operations of dental arch data generator 268 in generating jaw pair data based on incomplete jaw pair data, according to some embodiments. Input data 332 includes several missing teeth. Such data may be generated based on a jaw pair that is missing teeth, may be an artifact or result of incomplete or improper data collection or imaging methods, or the like. Input data 332 may include one or more partial teeth, e.g., damaged or broken teeth.

Input data 332 may be provided to dental arch data generator 268. Dental arch data generator 268 may use input data 332 to generate output data 334. Output data 334 may not resemble input data 332 by one or more metrics. For example, output data 334 may include data associated with one or more teeth of a jaw pair not represented in input data 332. Output data 334 may include complete teeth corresponding to broken or damaged teeth of input data 332.

FIG. 3G depicts example operations of dental arch data generator 268 in generating dental arch data based on incomplete dental arch data, according to some embodiments. Input data 336 may be provided to dental arch data generator 268. Similar to operations depicted in FIG. 3F, FIG. 3G depicts operations of dental arch generator 268 when input data 336 is incomplete, in the case of FIG. 3G, when input data 336 is an incomplete dental arch. In some embodiments, output data 338 may output a subset of jaw pair data indicated by a user, for example, only a target dental arch, one or more target teeth, or the like may be included in output data 338.

FIG. 3H depicts example operations of dental arch data generator 268 in generating dental arch data with different properties than input data 342, according to some embodiments. In some embodiments, input data 342 to dental arch data generator 268 may differ in one or more significant ways from output data 344. Input data 342 may be produced (or resemble data produced) via a different data collection method than output data 344. For example, input data 342 may be two-dimensional image data, as shown in FIG. 3H, and output data 344 may be three-dimensional model data that predicts three-dimensional properties of the same teeth, dental arches, jaw pair, or the like as input data 342. Data types, imaging types, data collection types, or the like may include two-dimensional imaging, three-dimensional imaging, point cloud scans, x-ray images, computed tomography images, or other measurement techniques.

FIG. 3I depicts example operations of dental arch data generator 268 in generating treatment predictions, according to some embodiments. Input data 346 to dental arch data generator 268 may include teeth that are misaligned, malocclusion, or other treatable properties. Dental arch data generator 268 may include one or more machine learning models configured to generate output data 348 after one or more stages of treatment. In some embodiments, output data 348 is a predicted representation of a jaw pair, one or more dental arches, or the like, after treatment is completed. In some embodiments, output data 348 is a predicted representation after one or more stages of treatment, such as in a treatment plan including several phases which progressively realign one or more teeth. Dental arch data generator 268 may include models for reducing dimensionality of jaw pair or dental arch data, transitioning the latent representation to a post-treatment representation, and expanding the compressed data to generate output data 348.

In the case of FIGS. 3J-M, possible expansions in the use of dental arch data generator 268 to include other oral structures, e.g., upper gingiva, lower gingiva, and palate, are discussed. FIG. 3J depicts example operations of dental arch data generator 268 in generating treatment predictions, according to some embodiments. Input data 370 to dental arch data generator 268 may include dental arch data, e.g., three-dimensional model data of teeth of an upper and lower dental arch. In some embodiments, input data 370 may include teeth that are misaligned, teeth exhibiting malocclusion, a dental arch missing one or more teeth to be restored (e.g., such as by providing a crown or implant), one or more teeth to be extracted, or the like. Dental arch data generator 268 may include one or more machine learning models configured to generate output data 372 after one or more stages of treatment. Output data 372 may include different structures of interest than input data 370. For example, output data 372 may be generated by extracting from a latent space representation, predictions including different oral structures than included in input data 370, e.g., including upper and lower gingiva, as depicted in FIG. 3J. Dental arch data generator 268 may include one or more models for reducing dimensionality of data (e.g., oral scan data), mapping the reduced order latent representation to post treatment, and extracting target predicted oral structure data, which may include different oral structures than those included in input data 370.

FIG. 3K depicts example operations of dental arch data generator 268 in generating oral structure predictions, according to some embodiments. Input data 374 to dental arch data generator 268 includes data indicative of a palate structure, e.g., the roof of the mouth, potentially including one or more of the hard palate and soft palate. Dental arch data generator 268 may include one or more machine learning models for generating output data 376. In some embodiments, a first set of oral structures (e.g., palate of input data 374) may be utilized to predict a different set of oral structures (e.g., dental arch of output data 376) associated with the first set of oral structures. Dental arch data generator 268 may include one or more trained machine learning models and/or other models configured to receive palate data as input data 374 and generate as output data 376 associated dental arch data.

FIG. 3L depicts example operations of dental arch data generator 268 in generating oral structure predictions, according to some embodiments. Input data 378 provided to dental arch data generator 268 includes dental arch data and gingiva data, e.g., includes a number of teeth as well as other oral structures. Further, the dental arch represented by input data 378 is missing a number of teeth. Dental arch data generator 268, based on the input data 378 (including tooth and gingiva data) may be configured to generate output data 380, indicative of a full dental arch (e.g., predictions of the jaw pair before tooth loss or after restorative treatment). In this example, dental arch data generator 268 includes one or more trained models configured to change which structures are represented (e.g., by including as input gingiva data, and optionally not including gingiva predictions in the output), as well as predict tooth positions different than the input data (e.g., after restorative treatment).

FIG. 3M depicts example operations of dental arch data generator 268 in predicting oral structures, according to some embodiments. Input data 382 includes dental arch data, e.g., data indicative of tooth shape and positions. Dental arch data generator 268 receives the input data 382. Dental arch data generator 268 includes one or more functions/processes (e.g., trained machine learning models) for generating output data in relation to input data. In the case of the example depicted in FIG. 3M, input data includes dental arch data of teeth, and output data 384 produced by dental arch data generator 268 includes gingiva data, e.g., without teeth included.

In some embodiments, operations such as those depicted in FIGS. 3E-M may be performed with any combination of input data segments or types, and target output data segments or types, and be within in the scope of this disclosure. For example, any combination of measurement techniques, oral structures, two-dimensional or three-dimensional data, etc., may be used as input to generate any combination of these features as output data, including adjustments to predicted structures in relation to decay, treatment, injury, and/or other changes of interest in dentition and/or oral structures of a jaw pair.

In some embodiments, one or more operations depicted in FIGS. 3E-M may be performed based on synthetic jaw pair data. For example, randomly sampled latent space representations may be decoded (e.g., by a decoder of dental arch data generator 268) to provide jaw pair data associated with the randomly sampled latent space locations. In another example, regions of the latent space associated with clusters of latent space representations (e.g., from input data associated with a number of dental arches) may be sampled to provide additional synthetic jaw pair data associated with the clusters. Clustering in the latent space may be performed, for example, by a modeling technique such as principle component analysis, machine learning clustering analysis, or the like. An unsupervised machine learning model may be utilized in performing clustering analysis in the latent space in some embodiments.

FIG. 3N depicts operations of a multi-stage treatment prediction platform 390, according to some embodiments. Input data 351 may be provided to first portion 320 (e.g., first portion 320 of FIG. 3A). A set of transformations 353 may be performed that adjust or remap latent space representations associated with input data 351. For example, transformations 353 may be performed by a set of models configured to predict the outcome of a phase or stage of treatment of one or more dental misalignments. The models may map input latent space vectors indicative of untreated or partially treated dental arches or dental arches to latent space vectors associated with the same dental arches after a phase of treatment. Any of the intermediate latent space vectors (e.g., between the first latent space vector representing input data 351 and some target end point vector that satisfies one or more treatment completion metrics) may be provided to a decoder. For example, while providing a treatment plan for a patient, after one of the transformations 353, the resulting latent space vector may be provided to decoder 355, and output data 357 indicative of a jaw pair after one stage of treatment may be generated. After a second of the transformations 353, the second resulting latent space vector may be provided to decoder 359 (which may be the same decoder as decoder 355) to generate output data 361, a third latent space vector may be provided to decoder 363 to generate output data 365, etc. While developing a treatment plan, a desired stopping point may be selected that balances desired results (e.g., desired level of alignment of a dental arch) with length of a treatment plan (e.g., a number of treatment stages required to reach the desired result).

FIGS. 4A-G are flow diagrams of methods 400A-G associated with training and utilizing models for predicting jaw pair data, according to certain embodiments. Methods 400A-G may be performed by processing logic that may include hardware (e.g., circuitry, dedicated logic, programmable logic, microcode, processing device, etc.), software (such as instructions run on a processing device, a general purpose computer system, or a dedicated machine), firmware, microcode, or a combination thereof. In some embodiment, methods 400A-G may be performed, in part, by predictive system 110 of FIG. 1. Method 400A may be performed, in part, by predictive system 110 (e.g., server machine 170 and data set generator 172 of FIG. 1. Predictive system 110 may use method 400A to generate a data set to at least one of train, validate, or test a machine learning model, in accordance with embodiments of the disclosure. Methods 400B-G may be performed by predictive server 112 (e.g., predictive component 114), client device 120, and/or server machine 180 (e.g., training, validating, and testing operations may be performed by server machine 180). In some embodiments, a non-transitory machine-readable storage medium stores instructions that when executed by a processing device (e.g., of predictive system 110, of server machine 180, of predictive server 112, etc.) cause the processing device to perform one or more of methods 400A-G.

For simplicity of explanation, methods 400A-G are depicted and described as a series of operations. However, operations in accordance with this disclosure can occur in various orders and/or concurrently and with other operations not presented and described herein. Furthermore, not all illustrated operations may be performed to implement methods 400A-G in accordance with the disclosed subject matter. In addition, those skilled in the art will understand and appreciate that methods 400A-G could alternatively be represented as a series of interrelated states via a state diagram or events.

FIG. 4A is a flow diagram of a method 400A for generating a data set for a machine learning model, according to some embodiments. Referring to FIG. 4A, in some embodiments, at block 401 the processing logic implementing method 400A initializes a training set T to an empty set.

At block 402, processing logic generates first data input (e.g., first training input, first validating input). The first data input may include data types related to an intended use of the machine learning model. The first data input may include jaw pair data for a machine learning model configured to map jaw pair data to a latent space, configured to compress jaw pair data. The first data input may include compressed data or latent space data for a machine learning model configured to map a first latent space vector to a second latent space vector. In some embodiments, the first data input may include a first set of features for types of data and a second data input may include a second set of features for types of data (e.g., as described with respect to FIG. 3B in segmented input data). Input data may include historical jaw pair data. Input data may include output data of another model. For example, a machine learning model configured to map from one latent space vector to a second latent space vector may take as input latent space vectors that are output of an encoder machine learning model.

In some embodiments, at block 403, processing logic optionally generates a first target output for one or more of the data inputs (e.g., first data input). In some embodiments, target output may represent an intended output space for the model. For example, a machine learning model configured to map a latent space jaw pair representation of a jaw pair before treatment to a predicted latent space jaw pair representation of the same jaw pair after treatment may be provided latent space representations of historical dental arches before and after treatment as training input and target output. As another example, a machine learning model configured to map a latent space representation of a jaw pair associated with a first measurement technique to a latent space representation of a jaw pair associated with a second jaw pair measurement technique may be provided with latent space representations of the jaw pair associated with each measurement technique as training input and target output. In some embodiments, no target output is generated (e.g., an unsupervised machine learning model capable of grouping or finding correlations in input data, rather than requiring target output to be provided). In one example, training input may at least in part be the same data as target output. For example, training an autoencoder may include reducing dimensionality of input data, reconstructing the input data to generate output data, and comparing at least a portion of the output data (e.g., teeth of a jaw pair that are represented in the input data) to the input data to determine updates to be performed to parameters of the autoencoder model.

At block 404, processing logic optionally generates mapping data that is indicative of an input/output mapping. The input/output mapping (or mapping data) may refer to the data input (e.g., one or more of the data inputs described herein), the target output for the data input, and an association between the data input(s) and the target output. In some embodiments, data segmentation may also be performed. For example, jaw pair data may be provided as target output, and may be associated via input/output mapping with one or more subsets of the jaw pair data (e.g., individual teeth, including identity, position, and orientation; groups of teeth; portions of a jaw; an arch; or a jaw of the jaw pair). In some embodiments, such as in association with machine learning models where no target output is provided, block 404 may not be executed.

At block 405, processing logic adds the mapping data generated at block 404 to data set T, in some embodiments.

At block 406, processing logic branches based on whether data set T is sufficient for at least one of training, validating, and/or testing a machine learning model, such as model 190 of FIG. 1. If so, execution proceeds to block 407, otherwise, execution continues back at block 402. It should be noted that in some embodiments, the sufficiency of data set T may be determined based simply on the number of inputs, mapped in some embodiments to outputs, in the data set, while in some other embodiments, the sufficiency of data set T may be determined based on one or more other criteria (e.g., a measure of diversity of the data examples, accuracy, etc.) in addition to, or instead of, the number of inputs.

At block 407, processing logic provides data set T (e.g., to server machine 180) to train, validate, and/or test machine learning model 190. In some embodiments, data set T is a training set and is provided to training engine 182 of server machine 180 to perform the training. In some embodiments, data set T is a validation set and is provided to validation engine 184 of server machine 180 to perform the validating. In some embodiments, data set T is a testing set and is provided to testing engine 186 of server machine 180 to perform the testing. In the case of a neural network, for example, input values of a given input/output mapping (e.g., numerical values associated with data inputs) are input to the neural network, and output values (e.g., numerical values associated with target outputs) of the input/output mapping are stored in the output nodes of the neural network. The connection weights in the neural network are then adjusted in accordance with a learning algorithm (e.g., back propagation, etc.), and the procedure is repeated for the other input/output mappings in data set T. After block 407, a model (e.g., model 190) can be at least one of trained using training engine 182 of server machine 180, validated using validating engine 184 of server machine 180, or tested using testing engine 186 of server machine 180. The trained model may be implemented by predictive component 114 (of predictive server 112) to generate predictive dental arch data 146 for performing signal processing, or for performing a corrective action.

FIG. 4B is a flow diagram of a method 400B for determining predictive jaw pair and/or dental arch data, according to some embodiments. At block 410, processing logic obtains first data of a dental arch, the first data of the dental arch having one or more first properties. The first data may be obtained from a jaw pair. The first data may have been measured from a jaw pair. The first data may have been obtained from one or more dental arches. The first data may be associated with a measurement technique, such as a two-dimensional image collection technique, three-dimensional modeling technique, an x-ray or computed tomography technique, or the like.

In some embodiments, a condition metric may indicate a property of the dental arch. For example, the condition metric may indicate which teeth are present in the dental arch, the dental arch data, or the like. As a further example, the condition metric may indicate that the jaw pair or dental arch exhibits one or more dental misalignments, dental malocclusions, or other orthodontic conditions. In some embodiments, the first value of the condition metric may indicate that the jaw pair exhibits a malocclusion. In some embodiments, a value of the condition metric may indicate a severity of a malocclusion or dental alignment. The data and/or dental arch may have properties including misalignments, presence or absence of teeth, data type, imaging type, data format, etc.

The first data may be or include three-dimensional model data, three-dimensional mesh data, point cloud data, two-dimensional image data (intraoral and/or external), two-dimensional measurement data (e.g., x-ray or computed tomography data), two-dimensional segmentation map data (e.g., planar partitions of three-dimensional data), or another type of data indicative of properties of one or more dental arch.

At block 412, processing logic processes the first data using one or more trained machine learning models. The one or more trained machine learning models generate a dimensionally reduced representation of the dental arch based on the first data and generate second data of the dental arch that has one or more second properties. The machine learning models may include an encoder. The machine learning models may include an encoder of an autoencoder model. The machine learning models may include one or more models for manipulating and/or mapping latent space data.

At block 414, processing logic obtains from the one or more machine learning models the second data of the dental arch that has one or more second properties. The second data is based on the dimensionally reduced representation. The one or more second properties are different from the one or more first properties. The second properties may indicate that the jaw pair or dental arch representation includes a different set of teeth from the first data, e.g., data associated with teeth missing from the first data may be included in the second data. The second properties may indicate that treatment has occurred, e.g., that a malocclusion has been treated. The second properties may indicate that a severity of a treatable jaw pair or dental arch condition has been reduced, e.g., the second properties may correspond to the jaw pair after one or more stages of treatment.

In some embodiments, generating the second data may include providing the dimensionally reduced representation to a second trained machine learning model. The second trained machine learning model may be configured to predict a transformed dimensionally reduced representation corresponding to the dental arch subsequent to treatment of a malocclusion. Generating the second data may further include providing the transformed dimensionally reduced representation to a third trained machine learning model, wherein the third trained machine learning model generates the second data based on the transformed dimensionally reduced representation. For example, the third trained machine learning model may be a decoder, configured to generate predictive dental arch data based on a latent space dental arch representation.

In some embodiments, one or more operations associated with generating second data may be performed according to a user selected parameter. For example, a user may select a desired output format or type, a desired model from a list of available trained machine learning models, a selection of portions of the dental arch data to generate or provide (e.g., a selection of teeth of interest), or the like.

In some embodiments, the dimensionally reduced representation may be provided to a second trained machine learning model. The second trained machine learning model may be configured to map the dimensionally reduced representation from a first latent space to a second latent space, to a second location within the first latent space, or the like. In some embodiments, the first latent space may be associated with a different type of dental arch or dental arch data than the second latent space. For example, the latent spaces may be associated with different jaw pair measurement techniques, the latent spaces may be associated with dental arches before and after orthodontic treatment, or the like. The method may further include providing the dimensionally reduced representation in the second latent space to a third trained machine learning model. The third trained machine learning model may output the second data, e.g., the third trained machine learning model may be a decoder configured to predict jaw pair data based on a latent space representation of a jaw pair.

At block 416, processing logic causes one or more representations of the dental arch to be displayed including the one based on the second data. In some embodiments, the images may be or include predictive images of a type different than the first data, such as two-dimensional images of a jaw pair (e.g., including two-dimensional segmentations of three-dimensional dental data), three-dimensional models of the jaw pair, x-ray or computed tomography images of the jaw pair (which may also be two-dimensional images, for example), or the like. In some embodiments, the images may be generated by a trained machine learning model configured to map latent space data to jaw pair data, e.g., a decoder. In some embodiments, the images may be based on output of a trained machine learning model configured to map latent space data to jaw pair data.

FIG. 4C is a flow diagram of a method 400C for generating predictive dental arch data, according to some embodiments. At block 420, processing logic obtains first data indicative of properties of a dental arch, the first data corresponding to a first imaging technique. The imaging technique may be a data collection technique for collecting jaw pair data, dental arch data, tooth data, etc. The imaging technique may be a jaw pair measurement technique, e.g., associated with a type of measurement equipment. The imaging technique may be a two-dimensional image collection technique. The first imaging technique may be a three-dimensional scanning technique. The first imaging technique may be an x-ray technique. The first imaging technique may be a computed tomography technique. The first imaging technique may be a cone-beam computed tomography technique. In some embodiments, the first data may be data from an intermediate stage of jaw pair data collection. For example, three-dimensional scanning of a jaw pair may occur in several stages. The first data may be a result of an incomplete scanning procedure, such as a point cloud of scan data, rather than a three-dimensional mesh or textured three-dimensional surface data. The first data may be predictive jaw pair data, e.g., output of a model for generating predictive jaw pair data.

At block 422, processing logic provides the first data to a first trained machine learning model as input, wherein the first trained machine learning model is configured to generate a dimensionally reduced representation of the dental arch based on the first data. The first trained machine learning model may be an encoder, e.g., of an autoencoder. The first trained machine learning model may be a hierarchical model, or another arrangement of multiple models. For example, the first trained machine learning model may be an encoder that encodes jaw pair and/or dental arch data in stages, with each stage including one or more encoders that operate on a portion of the input data.

At block 424, processing logic obtains second data indicative of properties of the dental arch. The second data is based on the dimensionally reduced representation. The second data corresponds to a second imaging technique. In some embodiments, the first data may be or comprise a point cloud (e.g., as generated by a three-dimensional scanning instrument) and the second data may include a three-dimensional model of the jaw pair.

In some embodiments, the dimensionally reduced representation of the dental arch may be provided to a model. The model may be a machine learning model, a rule-based model, or the like. The model may be configured to map the dimensionally reduced representation of the dental arch from a first latent space to a second latent space. The model may be configured to map the dimensionally reduced representation of the dental arch from a first location in the first latent space to a second location in the first latent space. The dimensionally reduced representation of the dental arch, expressed in the second latent space or the second location in the first latent space, may then be provided to a second machine learning model. The second machine learning model may be a decoder. The second machine learning model generates output, which may include the second data. For example, the second machine learning model may be configured to map latent space data to predictive jaw pair data. In some embodiments, data of a complete jaw pair (e.g., including an upper dental arch and a lower dental arch) may be utilized for any of the methods or operations described as including data of a dental arch. In some embodiments, the first data may be of a dental arch, and the second data may be or include data of a second dental arch. For example, the first data may be of a lower dental arch, and predictive data may be generated of a corresponding upper dental arch.

At block 426, processing logic causes one or more images indicative of the second data to be displayed. Processing logic may cause the second data to be displayed. In some embodiments, the second data may be provided to further applications. For example, the second data may be predictive three-dimensional jaw pair model data, which may be provided to any application that receives three-dimensional jaw pair data as input. For example, treatment prediction models, treatment output prediction models, jaw articulation models, or the like may be provided output of a latent space jaw pair machine learning model as input. In some embodiments, a latent space representation of jaw pairs and/or dental arches may be utilized to track treatment progress, e.g., by providing predicted jaw pair three-dimensional models based on two-dimensional image data of a jaw pair undergoing treatment. In some embodiments, predictive jaw pair data may be further augmented, such as by providing predicted color or texture data as output of a machine learning model decoder. For example, predictions based on input data that do not include color information may be augmented with a predicted texture map (e.g., a map that applies color data to a three-dimensional model).

FIG. 4D is a flow diagram of a method 400D for training a machine learning model for reducing dimensionality of jaw pair and/or dental arch data, according to some embodiments. At block 430, processing logic obtains first data of a dental arch having one or more first properties. In some embodiments, the properties may describe a condition of the dental arch, e.g., malocclusion, missing teeth, etc. In some embodiments, the properties may describe a condition of the data, e.g., two-dimensional image data, data format, imaging technique, etc. The first data may share one or more features with jaw pair data discussed in connection with FIGS. 4A-C.

At block 432, processing logic trains a first machine learning model based on the first data. Training the first machine learning model includes performing a number of operations. At block 434, processing logic causes the first machine learning model to obtain the first data. The first data may include training input. The first data may include target output. The first data may include input/output mapping.

At block 436, processing logic causes the first machine learning model to reduce dimensionality of the first data to generate first compressed data. There may be loss functions imposed on the compression to ensure an adequate compression is achieved (e.g., a level of data compression satisfying a target threshold condition).

Reduction of dimensionality to generate compressed data may be performed by an encoder, e.g., of an autoencoder.

At block 438, the first machine learning model generates first reconstructed data based on the first compressed data. Generating the first reconstructed data may be performed by a decoder, e.g., of an autoencoder.

At block 440, the first machine learning model causes parameters of the first machine learning model to be adjusted based on one or more differences between the first data and the first reconstructed data. Training operations may be repeated until training is completed, e.g., until a target accuracy of the model is achieved.

In some embodiments, a second machine learning model may be trained. The second machine learning model may be configured to accept as input compressed data. The second machine learning model may be configured to generate as output compressed data. Training the second machine learning model may include providing first dimensionally reduced data associated with a historical jaw pair and/or dental arch data having a first characteristic to the second machine learning model as training input. Training the second machine learning model may further include providing second dimensionally reduced data associated with the historical jaw pair having a second characteristic to the second machine learning model as target output. The second machine learning model may be trained based on the training input and the target output. The second machine learning model may be trained to map jaw pair data (in a compressed latent space form) from dental arches having the first characteristic to dental arches having the second characteristic. For example, the characteristic may be indicative of a malocclusion, and the second machine learning model may be configured to provide a mapping between latent space representations of dental arches before malocclusion treatment to dental arches after malocclusion treatment.

In some embodiments, a second machine learning model may be trained that is configured to map dimensionally reduced data from a first latent space to a second latent space (e.g., first latent vector space to a second latent vector space). Training the second machine learning model may include providing first dimensionally reduced data associated with a historical jaw pair or dental arch in the first latent space to the second machine learning model as training input. The first dimensionally reduced data may be output from a machine learning model, such as an encoder. Training the second machine learning model may further include providing second dimensionally reduced data associated with the historical jaw pair or dental arch in the second latent space to the second machine learning model as target output. The second dimensionally reduced data may be or include output of a third machine learning model configured to dimensionally reduce jaw pair data or dental arch data. In some embodiments, different latent spaces may be utilized for different tasks. For example, different latent spaces may be utilized for teeth with different qualities, for different jaw pair data generation techniques, for different jaw pair measurement techniques, or the like.

FIG. 4E is a flow diagram of a method 400E for determining predictive oral cavity data, according to some embodiments. Methods, procedures, and operations of method 400E may share one or more features with method 400B of FIG. 4B. At block 450, process logic obtains first data of an oral cavity. The first data may be captured, for example, at a dental practice using an intraoral scanner, and may be uploaded to a server computing device that may execute one or more operations of method 400E in embodiments. In another example, the first data may be captured by a mobile device (e.g., a mobile phone, tablet computer, etc.) of a user and may be uploaded from the mobile device to a server computing device that may execute one or more operations of method 400E in embodiments. The first data of the oral cavity has first one or more properties. The first one or more properties of the first data may reflect realities of the oral cavity. For example, the first one or more properties may include presence or absence of one or more teeth in the oral cavity, alignment or position of one or more teeth, presence of evidence of one or more dental disorders (e.g., malocclusion), or the like. The first one or more properties may include context of the oral cavity, e.g., an indication of dental or orthodontic treatment that has been performed or is to be performed with respect to the oral cavity, such as one or more stages of orthodontic treatment. The first one or more properties may reflect one or more aspects of the data, including quality of data, type of data (e.g., two-dimensional image data, three-dimensional model data, etc.), associated measurement technique (e.g., intraoral scan data, x-ray data, etc.), and/or another property of the data.

At block 452, process logic processes the first data using one or more trained machine learning models. The one or more trained machine learning models generate a dimensionally reduced representation of the oral cavity based on the first data, and further generate second data of the oral cavity that has one or more second properties. In some embodiments, an encoder, decoder, or autoencoder may be included in the one or more trained machine learning models. The one or more trained machine learning models may include models for mapping from data including first properties to data including second properties within a latent space, for mapping between latent spaces, or the like for performance of various operations in connection with the first data.

Process logic obtains from the one or more trained machine learning models the second data of the oral cavity, including the one or more second properties. The second properties are different than the first, e.g., the output data is different in some way than the input data (e.g., indicative of pre- or post-treatment dentition, associated with different measurement methods, etc.). The second data is based on the dimensionally reduced representation.

The one or more operations associated with generating the second data may be performed according to user selection of one or more parameters, e.g., user selection of the second properties, to target output data of interest. In some embodiments, the dimensionally reduced representation may be provided to a trained machine learning model (e.g., of the one or more trained machine learning models) that may map the dimensionally reduced representation to a different location of the latent space or a different latent space. For example, the one or more models may include a model for generating the dimensionally reduced representation, a model for mapping the dimensionally reduced representation to a different location of the latent space (e.g., representing adjustments to the oral cavity such as treatment), and another model for extracting the second data from the remapped dimensionally reduced representation.

At block 456, process logic causes a representation of the oral cavity to be displayed based on the second data. In some embodiments, the representation may include an image, e.g., a projection or isometric view of a three-dimensional model, a two-dimensional image (e.g., of a predicted patient smile), a predicted x-ray or other imaging technique data, etc. In an embodiment, the representation of the oral cavity is transmitted to a computing device of a dental practice (e.g., of a doctor) and/or of a patient (e.g., to a user device of a patient) for display thereon.

At block 458, processing logic may store the second data in a data store.

FIG. 4F is a flow diagram of a method 400F for generating predictive oral cavity data, according to some embodiments. Processes and operations of method 400F may share one or more features with method 400C of FIG. 4C. At block 460, process logic obtains first data indicative of properties of a oral cavity. The first data may correspond to a first imaging technique. In some embodiments, the first data may relate to an incomplete measurement or sparse measurement technique, e.g., a sparse point cloud, an incomplete intraoral scan, or the like.

At block 462, process logic provides the first data to a first trained machine learning model, wherein the first trained machine learning model processes the first data and generates a dimensionally reduced representation of the oral cavity based on the first data. The first trained machine learning model may be an encoder, e.g., the encoding portion of an autoencoder.

At block 464, process logic obtains or generates second data indicative of properties of the oral cavity. The second data is based on the dimensionally reduced representation. The second data corresponds to a second imaging technique, different than the first imaging technique. In some embodiments, the first data may be or comprise a point cloud (e.g., generated by an intraoral scanning instrument). In some embodiments, the second data may include a three-dimensional model of the oral cavity, e.g., may resemble an intraoral scan.

In some embodiments, the dimensionally reduced representation may be provided to a model (e.g., a second model of the one or more models). The model may be a machine learning model, a rule-based model, or the like. The model may be configured to map the dimensionally reduced representation of the dental arch from a first latent space to a second latent space, e.g., two latent spaces associated with different measurement techniques, different types of images (e.g., images of a smile or images of dentition), or the like. The model may be configured to map the dimensionally reduced representation of the dental arch from a first location in the first latent space corresponding to an oral cavity with first properties (e.g., tooth placement and shape) to a second location in the second latent space corresponding to an oral cavity with the same properties or different properties, as appropriate for target operations.

In some embodiments, additional models (e.g., numerical optimization models, trained machine learning models, etc.) may act on the dimensionally reduced data, e.g., to map the dimensionally reduced data to other locations of a latent space, to locations of a different latent space, or the like.

At block 468, process logic causes the second data to be displayed, e.g., causes a visual representation of the oral cavity based on the second data to be displayed, such as via a graphical user interface.

FIG. 4G is a flow diagram of a method 400G for training a machine learning model for reducing dimensionality of oral cavity data, according to some embodiments. Operations described with respect to method 400G may share one or more features with method 400D of FIG. 4D, in some embodiments. At block 470, process logic obtains first data of an oral cavity having one or more first properties. In some embodiments, the properties may include or describe conditions of the oral cavity, conditions of the data, a measurement means for obtaining the data or that the data resembles, or the like. Further, a large amount of data associated with a number of oral cavities may be similarly obtained and processed for enacting training operations of the machine learning model.

At block 472, process logic trains a first machine learning model based on the training data. Training the first machine learning model includes performing several operations. At block 474, process logic causes the first machine learning model to obtain the first data, e.g., the first data is provided to the machine learning model, the machine learning model receives the first data, etc. The first data may include training input. The first data may include target output. The first data may include input/output mapping data.

At block 476, process logic reduces dimensionality of the first data to generate first compressed data. There may be loss functions imposed on the compression to ensure adequate compression is achieved. Dimensionality reduction (e.g., projection to a latent space) may be performed by an encoder, e.g., of an autoencoder.

At block 478, the first machine learning model generates first reconstructed data based on the first compressed data. Generating the first reconstructed data may be performed by a decoder.

At block 480, the first machine learning model causes parameters of the first machine learning model to be adjusted based on one or more differences between the first data and the first reconstructed data. Training operations may be continued until a target accuracy of the model is achieved.

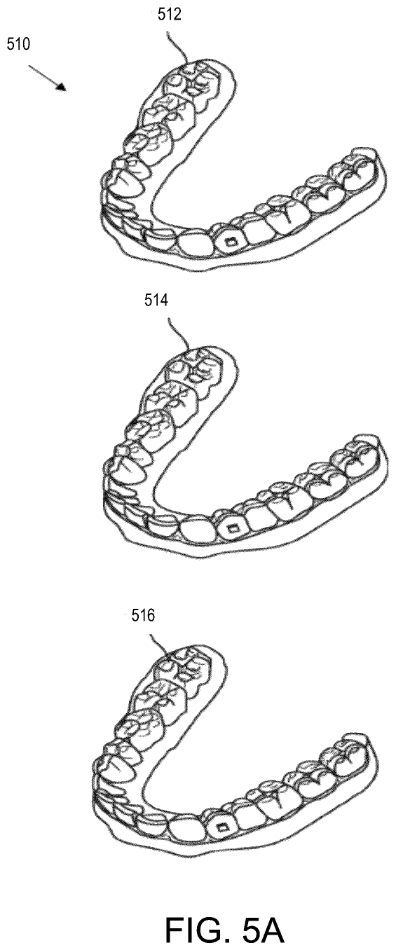

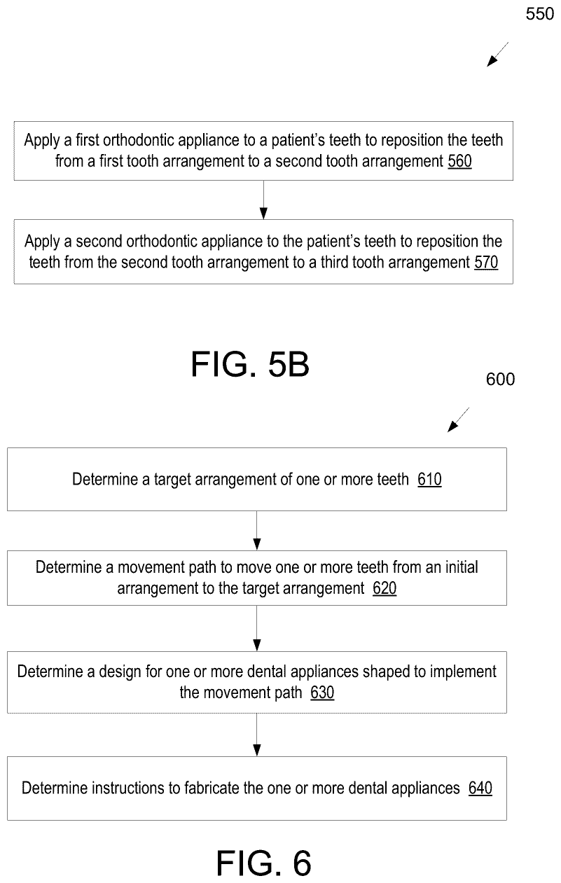

FIG. 5A illustrates a tooth repositioning system 510 including a plurality of appliances 512, 514, 516. The appliances 512, 514, 516 can be designed based on generation of a sequence of 3D models of dental arches, which may be generated according to the techniques discussed herein above. Any of the appliances described herein can be designed and/or provided as part of a set of a plurality of appliances used in a tooth repositioning system, and may be designed in accordance with an orthodontic treatment plan generated in accordance with embodiments of the present disclosure. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 510 can include a first appliance 512 corresponding to an initial tooth arrangement, one or more intermediate appliances 514 corresponding to one or more intermediate arrangements, and a final appliance 516 corresponding to a target arrangement. A target tooth arrangement can be a planned final tooth arrangement selected for the patient's teeth at the end of all planned orthodontic treatment, as optionally output using a trained machine learning model. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 512, 514, 516 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

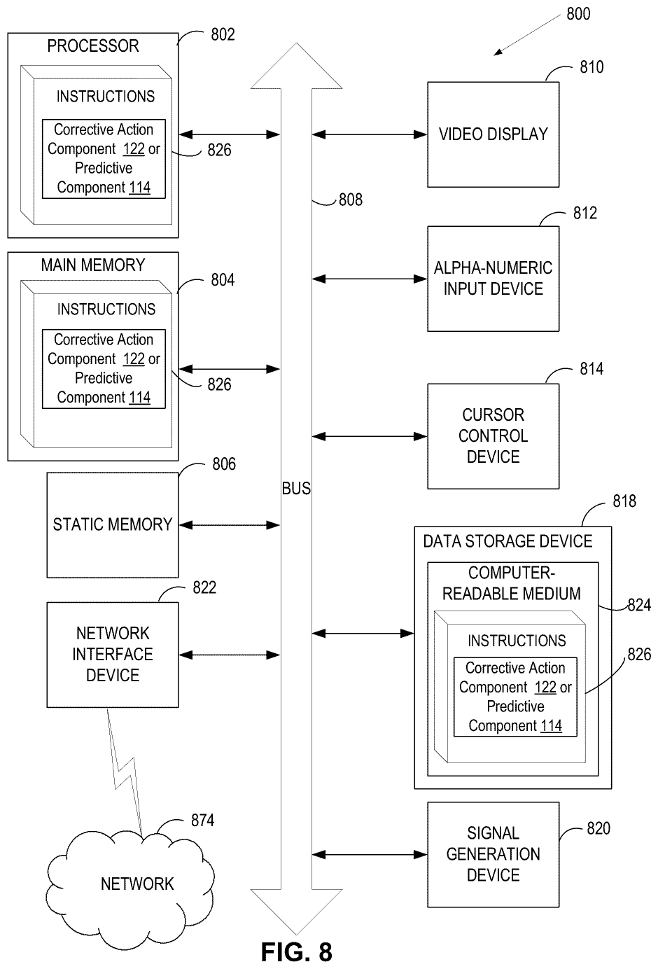

In an example of indirect fabrication, a mold of a patient's dental arch may be fabricated from a digital model of the dental arch generated by a trained machine learning model as described above, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 512, 514, 516 after the digital models of the appliances 512, 514, 516 have been processed by processing logic of a computing device, such as the computing device in FIG. 8. The processing logic may include hardware (e.g., circuitry, dedicated logic, programmable logic, microcode, etc.), software (e.g., instructions executed by a processing device), firmware, or a combination thereof. For example, one or more operations may be performed by a processing device executing dental arch data generator 268 of FIG. 2.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images or scans to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner may determine a desired treatment outcome, which includes final positions and orientations for the patient's teeth. In one embodiment, dental arch data generator 268 outputs a desired treatment outcome based on processing the virtual 3D model of the patient's dental arch (or other dental arch data associated with the virtual 3D model). Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. In one embodiment, for each treatment stage dental arch data generator 268 outputs a different 3D model of the dental arch. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances, stereolithography (SLA), also known as optical fabrication solid imaging, is used to fabricate an SLA mold. In SLA, the mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the mold. The platform is lowered incrementally, and the light source traces a new pattern over the platform to form another layer of the mold at each increment. This process repeats until the mold is completely fabricated. Once all of the layers of the mold are formed, the mold may be cleaned and cured.

Materials such as a polyester, a co-polyester, a polycarbonate, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold. The materials used for fabrication of the mold can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 512, 514, 516 can be pressure formed or thermoformed over the molds. Each mold may be used to fabricate an appliance that will apply forces to the patient's teeth at a particular stage of the orthodontic treatment. The appliances 512, 514, 516 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

Additional information may be added to the appliance. The additional information may be any information that pertains to the appliance. Examples of such additional information includes a part number identifier, patient name, a patient identifier, a case number, a sequence identifier (e.g., indicating which appliance a particular liner is in a treatment sequence), a date of manufacture, a clinician name, a logo and so forth. For example, after determining there is a probable point of damage in a digital design of an appliance, an indicator may be inserted into the digital design of the appliance. The indicator may represent a recommended place to begin removing the polymeric appliance to prevent the point of damage from manifesting during removal in some embodiments. In embodiments, the additional information may be automatically added to a generated 3D model by dental arch data generator 268 in generation of the 3D model.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. In one embodiment, dental arch data generator 268 outputs a cutline for an appliance associated with a 3D model output by the dental arch generator 268. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

FIG. 5B illustrates a method 550 of orthodontic treatment using a plurality of appliances, in accordance with embodiments. The method 550 can be practiced using any of the appliances or appliance sets described herein. In block 560, a first orthodontic appliance is applied to a patient's teeth in order to reposition the teeth from a first tooth arrangement to a second tooth arrangement. In block 570, a second orthodontic appliance is applied to the patient's teeth in order to reposition the teeth from the second tooth arrangement to a third tooth arrangement. The method 550 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 6 illustrates a method 600 for designing an orthodontic appliance to be produced by direct or indirect fabrication, in accordance with embodiments. The method 600 can be applied to any embodiment of the orthodontic appliances described herein, and may be performed using one or more trained machine learning models in embodiments. Some or all of the blocks of the method 600 can be performed by any suitable data processing system or device, e.g., one or more processors configured with suitable instructions.

At block 610 a target arrangement of one or more teeth of a patient may be determined. The target arrangement of the teeth (e.g., a desired and intended end result of orthodontic treatment) can be received from a clinician in the form of a prescription, can be calculated from basic orthodontic principles, can be extrapolated computationally from a clinical prescription, and/or can be generated by a trained machine learning model such as treatment dental arch data generator 268 of FIG. 2. With a specification of the desired final positions of the teeth and a digital representation of the teeth themselves, the final position and surface geometry of each tooth can be specified to form a complete model of the tooth arrangement at the desired end of treatment.

In block 620, a movement path to move the one or more teeth from an initial arrangement to the target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. An initial arrangement may be estimated by projecting some measurement of the patient's teeth to a latent space, and obtaining from the latent space a representation of the initial arrangement. From the obtained data, a digital data set such as a 3D model of the patient's dental arch or arches can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, optionally including measured or extrapolated hidden surfaces and root structures, as well as surrounding bone and soft tissue.

Having both an initial position and a target position for each tooth, a movement path can be defined for the motion of each tooth. Determining the movement path for one or more teeth may include identifying a plurality of incremental arrangements of the one or more teeth to implement the movement path. In some embodiments, the movement path implements one or more force systems on the one or more teeth (e.g., as described below). In some embodiments, movement paths are determined by a trained machine learning model such as treatment plan generator 276. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In some embodiments, a force system to produce movement of the one or more teeth along the movement path is determined. In one embodiment, the force system is determined by a trained machine learning model. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be considered. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 630, a design for one or more dental appliances shaped to implement the movement path is determined. In one embodiment, the one or more dental appliances are shaped to move the one or more teeth toward corresponding incremental arrangements. In some embodiments, results of one or more stages of treatment may be predicted by dental arch data generator 268. Determination of the one or more dental or orthodontic appliances, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systemes of Waltham, MA.

In block 640, instructions for fabrication of the one or more dental appliances are determined or identified. In some embodiments, the instructions identify one or more geometries of the one or more dental appliances. In some embodiments, the instructions identify slices to make layers of the one or more dental appliances with a 3D printer. In some embodiments, the instructions identify one or more geometries of molds usable to indirectly fabricate the one or more dental appliances (e.g., by thermoforming plastic sheets over the 3D printed molds). The dental appliances may include one or more of aligners (e.g., orthodontic aligners), retainers, incremental palatal expanders, attachment templates, and so on.

In one embodiment, instructions for fabrication of the one or more dental appliances are generated by a trained model. In some embodiments, predictions of treatment progression and/or treatment appliances may be performed and/or aided by dental arch data generator 268. The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by 3D printing a mold and thermoforming a plastic sheet over the mold.

Method 600 may comprise additional blocks: 1) The upper arch and palate of the patient is scanned intraorally to generate three dimensional data of the palate and upper arch; 2) The three dimensional shape profile of the appliance is determined to provide a gap and teeth engagement structures as described herein.

Although the above blocks show a method 600 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 600 may be performed with any suitable fabrication system or device, such as the embodiments described herein. Some of the blocks may be optional, and the order of the blocks can be varied as desired.

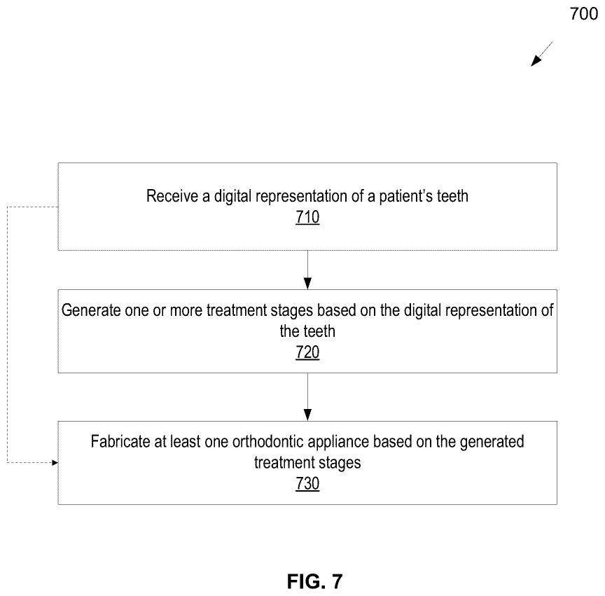

FIG. 7 illustrates a method 700 for digitally planning an orthodontic treatment and/or design or fabrication of an appliance, in accordance with embodiments. The method 700 can be applied to any of the treatment procedures described herein and can be performed by any suitable data processing system.

In block 710, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 720, one or more treatment stages are generated based on the digital representation of the teeth. In some embodiments, the one or more treatment stages are generated based on processing of input dental arch data by a trained machine learning model such as dental arch data generator 268. Each treatment stage may include a generated 3D model of a dental arch at that treatment stage. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 730, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired. The fabrication of the appliance may include automated removal of the appliance from a mold (e.g., automated removal of an untrimmed shell from mold a using a shell removal device).

In some instances, staging of various arrangements or treatment stages may not be necessary for design and/or fabrication of an appliance. As illustrated by the dashed line in FIG. 7, design and/or fabrication of an orthodontic appliance, and perhaps a particular orthodontic treatment, may include use of a representation of the patient's teeth (e.g., receive a digital representation of the patient's teeth at block 710), followed by design and/or fabrication of an orthodontic appliance based on a representation of the patient's teeth in the arrangement represented by the received representation.

FIG. 8 is a block diagram illustrating a computer system 800, according to some embodiments. In some embodiments, computer system 800 may be connected (e.g., via a network, such as a Local Area Network (LAN), an intranet, an extranet, or the Internet) to other computer systems. Computer system 800 may operate in the capacity of a server or a client computer in a client-server environment, or as a peer computer in a peer-to-peer or distributed network environment. Computer system 800 may be provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 800 may include a processing device 802, a volatile memory 804 (e.g., Random Access Memory (RAM)), a non-volatile memory 806 (e.g., Read-Only Memory (ROM) or Electrically-Erasable Programmable ROM (EEPROM)), and a data storage device 818, which may communicate with each other via a bus 808.

Processing device 802 may be provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor.

Computer system 800 may further include a network interface device 822 (e.g., coupled to network 874). Computer system 800 also may include a video display unit 810 (e.g., an LCD), an alphanumeric input device 812 (e.g., a keyboard), a cursor control device 814 (e.g., a mouse), and a signal generation device 820.

In some embodiments, data storage device 818 may include a non-transitory computer-readable storage medium 824 (e.g., non-transitory machine-readable medium) on which may store instructions 826 encoding any one or more of the methods or functions described herein, including instructions encoding components of FIG. 1 (e.g., predictive component 114, corrective action component 122, model 190, etc.) and for implementing methods described herein.

Instructions 826 may also reside, completely or partially, within volatile memory 804 and/or within processing device 802 during execution thereof by computer system 800, hence, volatile memory 804 and processing device 802 may also constitute machine-readable storage media.

While computer-readable storage medium 824 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “performing,” “providing,” “obtaining,” “causing,” “accessing,” “determining,” “adding,” “using,” “training,” “reducing,” “generating,” “correcting,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and embodiments, it will be recognized that the present disclosure is not limited to the examples and embodiments described. The scope of the disclosure should be determined with reference to the following claims, along with the full scope of equivalents to which the claims are entitled.

1. A method, comprising:
obtaining first data of a dental arch, the first data of the dental arch having one or more first properties;
processing the first data using one or more trained machine learning models, wherein the one or more trained machine learning models generate a dimensionally reduced representation of the dental arch based on the first data and generate second data of the dental arch that has one or more second properties, wherein the second data is based on the dimensionally reduced representation, and wherein the one or more second properties are different from the one or more first properties; and
causing a representation of the dental arch to be displayed based on the second data.

2. The method of claim 1, wherein the first data comprises one or more of:
one or more two-dimensional images of the dental arch;
one or more three-dimensional models of the dental arch;
one or more three-dimensional models of one or more teeth associated with the dental arch; or
one or more computed tomography (CT) images of the dental arch.

3. The method of claim 1, wherein the one or more first properties comprise at least a partial absence of one or more teeth in the dental arch, and wherein the second data comprises the one or more teeth that are not included in the first data.

4. The method of claim 1, wherein the one or more first properties are of one or more teeth of the dental arch, and wherein the one or more second properties are of the one or more teeth of the dental arch.

5. The method of claim 4, wherein the one or more first properties comprise a presence of one or more dental malocclusions for the one or more teeth, and wherein the one or more second properties comprise an absence of the one or more dental malocclusions for the one or more teeth.

6. The method of claim 4, wherein the one or more first properties comprise a first severity of a malocclusion for the one or more teeth, and the one or more second properties comprise a second severity of the malocclusion.

7. The method of claim 6, wherein the second severity of the malocclusion comprises a prediction of a condition of the one or more teeth after a stage of orthodontic treatment.

8. The method of claim 7, wherein the one or more trained machine learning models further generates third data of the dental arch having one or more third properties, wherein the one or more third properties comprise a third severity of the malocclusion after an additional stage of the orthodontic treatment.

9. The method of claim 1, wherein the dimensionally reduced representation is generated by a first trained machine learning model of the one or more trained machine learning models and the second data is generated by a second trained machine learning model of the one or more trained machine learning models based on the dimensionally reduced representation.

10. The method of claim 1, wherein:
the dimensionally reduced representation is generated by a first trained machine learning model of the one or more trained machine learning models;
a second trained machine learning model of the one or more trained machine learning models generates a transformed dimensionally reduced representation corresponding to the dental arch based on the dimensionally reduced representation; and
a third trained machine learning model of the one or more trained machine learning models generates the second data based on the transformed dimensionally reduced representation.

11. The method of claim 1, wherein:
the dimensionally reduced representation is generated by a first trained machine learning model of the one or more trained machine learning models;
a numerical optimization model generates a transformed dimensionally reduced representation corresponding to the dental arch based on the dimensionally reduced representation; and
a second trained machine learning model of the one or more trained machine learning models generates the second data based on the transformed dimensionally reduced representation.

12. The method of claim 1, further comprising receiving a user selection of one or more teeth, wherein the second data comprises a three-dimensional model comprising models of the one or more teeth of the user selection.

13. The method of claim 1, wherein the one or more trained machine learning models comprise a first trained machine learning model that generates the dimensionally reduced representation, a second trained machine learning model that processes the dimensionally reduced representation, and a third trained machine learning model, the method further comprising:
providing the dimensionally reduced representation to the second trained machine learning model, wherein the second trained machine learning model maps the dimensionally reduced representation from a first latent space to a second latent space; and
providing the dimensionally reduced representation in the second latent space to a third trained machine learning model, wherein output from the third trained machine learning model comprises the second data.

14. The method of claim 13, wherein the first latent space is associated with a first dental arch data generation technique, and wherein the second latent space is associated with a second dental arch data generation technique, different from the first.

15. The method of claim 1, wherein the first data is two-dimensional data and wherein the second data is three-dimensional data.

16. The method of claim 1, further comprising obtaining third data of an associated dental arch from the one or more machine learning models, wherein the dental arch is a first dental arch of either an upper dental arch or a lower dental arch of a dental arch pair, and wherein the associated dental arch is the other of the upper dental arch or the lower dental arch of the dental arch pair.

17. The method of claim 1, wherein the one or more trained machine learning models comprises an encoder model, and wherein the encoder model comprises a plurality of encoder models, wherein one or more encoder models of the plurality of encoder models are provided a segment of the first data.

18. The method of claim 17, wherein the plurality of encoder models comprises:
a first set of encoder models, wherein each encoder model of the first set of encoder models is configured to receive input data associated with a target tooth; and
a second set of encoder models, wherein each encoder model of the second set of encoder models is configured to receive input data associated with either an upper or lower dental arch.

19. The method of claim 18, wherein the plurality of encoder models further comprises a first encoder model configured to receive input data associated with both the upper and lower dental arch.

20. The method of claim 17, wherein the one or more trained machine learning models further comprises a decoder model, and wherein the decoder model comprises a plurality of decoder models, wherein each of the plurality of decoder models corresponds to one or the plurality of encoder models.

21. A method, comprising:
obtaining first data of a dental arch, the first data corresponding to a first imaging technique;
providing the first data to a first trained machine learning model, wherein the first trained machine learning model generates a dimensionally reduced representation of the dental arch based on the first data;
obtaining second data of the dental arch, wherein the second data is based on the dimensionally reduced representation, and wherein the second data corresponds to a second imaging technique; and
causing the second data to be displayed.

22. The method of claim 21, wherein the first imaging technique comprises:
two-dimensional image collection;
three-dimensional intraoral scanning;
two-dimensional image segmentation; or
computed tomography.

23. The method of claim 21, wherein the first data comprises a point cloud, and wherein the second data comprises a three-dimensional model of the dental arch.

24. The method of claim 21, further comprising:
providing the dimensionally reduced representation of the dental arch to a model configured to map the dimensionally reduced representation of the dental arch from a first latent space to a second latent space; and
providing the dimensionally reduced representation of the dental arch in the second latent space to a second trained machine learning model, wherein the second data comprises output of the second trained machine learning model based on the dimensionally reduced representation of the dental arch in the second latent space.

25-66. (canceled)

67. A system, comprising memory and a processing device coupled to the memory, wherein the processing device is configured to:
obtain first data of an oral cavity, the first data having one or more first properties;
process the first data using one or more trained machine learning models, wherein the one or more trained machine learning models generate a dimensionally reduced representation of the oral cavity based on the first data, and generate second data of the oral cavity that has one or more second properties, wherein the second data is based on the dimensionally reduced representation, and wherein the one or more second properties are different from the one or more first properties; and
cause a representation of the oral cavity to be displayed based on the second data.

68. A non-transitory, machine-readable storage medium storing instructions which, when executed, cause a processing device to perform operations comprising:
obtaining first data of an oral cavity, the first data having one or more first properties;
processing the first data using one or more trained machine learning models, wherein the one or more trained machine learning models generate a dimensionally reduced representation of the oral cavity based on the first data, and generate second data of the oral cavity that has one or more second properties, wherein the second data is based on the dimensionally reduced representation, and wherein the one or more second properties are different from the one or more first properties; and
causing a representation of the oral cavity to be displayed based on the second data.
